(19) United States
(12) Patent Application Publication
(10) Pub. No.: US 2024/0350619 A1
(43) Pub. Date: Oct. 24, 2024

(54) VACCINES AND COMPOSITIONS BASED ON SARS-COV-2 S PROTEIN

(71) Applicants: GUANGZHOU RIBOBIO CO., LTD., Guangzhou, Guangdong (CN); ARGORNA PHARMACEUTICALS CO., LTD., Guangzhou, Guangdong (CN)

(72) Inventors: Bill Biliang ZHANG, Boston, MA (US); Lin MA, Guangzhou, Guangdong (CN); Jian WEN, Guangzhou, Guangdong (CN); Hong ZHANG, Guangzhou, Guangdong (CN); Huiling ZHONG, Guangzhou, Guangdong (CN)

(21) Appl. No.: 18/027,132

(22) PCT Filed: May 10, 2022
(86) PCT No.: PCT/CN2022/091986

§ 371 Date: Mar. 20, 2023

(30) Foreign Application Priority Data
Jan. 10, 2022 (CN) 202210019169.6

(51) Int. Cl. A61K 39/215; A61K 39/00; A61P 31/14; C07K 14/005; C12N 7/00; C12N 15/11; C12N 15/85

(52) U.S. Cl. CPC A61K 39/215; A61P 31/14; C07K 14/005; C12N 7/00; C12N 15/11; C12N 15/85; A61K 2039/53; A61K 2039/55555; A61K 2039/70; C12N 2770/20022; C12N 2770/20034; C12N 2830/50

(57) ABSTRACT

This disclosure provides vaccines and compositions based on SARS-COV-2 S protein, and specifically relates to recombinant SARS-COV-2 spike protein (Sprotein) and mRNA and DNA coding thereof. This disclosure also relates to recombinant plasmid comprising DNA sequence encoding recombinant S protein. This disclosure further relates to composition comprising the recombinant S protein and/or mRNA mentioned above, mRNA-carrier particle such as lipid nanoparticle (LNP), and composition such as a vaccine composition.

### CROSS-REFERENCE TO RELATED APPLICATIONS

This application is the national stage entry of PCT/CN2022/091986, filed on May 10, 2022, which claims the benefit of priority to Chinese Patent Application No, 202210019169.6 filed on Jan. 10, 2022, which are incorporated by reference in their entirety herein.

### SEQUENCE LISTING

The instant application contains a Sequence Listing which has been submitted electronically in ASCII format and is hereby incorporated by reference in its entirety. Said ASCII copy is named PN189329_Sequence_listing.txt and is 312,697 bytes in size, and contains a sequence listing identical to the sequence listing filed in the corresponding international application No, PCT/CN2022/091986, filed on May 10, 2022.

### TECHNICAL FIELD

This disclosure belongs to the technical field of biomedicine and vaccine, especially relates to vaccines and compositions against SARS-COV-2, such as SARS-COV-2 Delta variant (B.1.617.2) and Omicron variant (B.1.1.529).

### BACKGROUND ART

The genome of SARS-COV-2 mutates constantly with the spread in different host groups, generating a variety of subtypes, wherein SARS-COV-2 Delta variant, B1.617.2, is a new variant first reported in India; SARS-COV-2 Omicron variant (B.1.1.529) is another highly infectious variant first found in South Africa. There is no mature special medicine can cure SARS-COV-2 Delta variant and SARS-COV-2 Omicron variant now, and effective vaccine is urgently needed.

Compared with SARS-COV-2 reported in early stage, the genome of SARS-COV-2 Delta variant occurs mutation in multiple positions of the genome. These mutations trigger coronavirus immune escape, resulting in stronger human adaptability, faster spreading speed, higher viral load, longer treatment period, easier developing into severe disease and other characteristics in the viruses, compared with other early novel coronavirus subtypes.

Compared with SARS-COV-2 reported in early stage, the genome of SARS-COV-2 Omicron variant also mutates in multiple positions, including mutations occurred in S protein, ORF1a, ORF1b, ORF9b, M protein, E protein and N protein. These mutations not only result in strengthening the Omicron variant's spread ability, but also in enhancing this viral subtype's resistance ability against antibody's protective effect, making it more resistant to the current SARS-COV-2 vaccine and be able to escape from the immune response induced by vaccine. Thus, developing corresponding targeted vaccine is urgently needed.

As the 3^{rd} generation vaccine, mRNA vaccine can induce body to produce humoral immunity and cellular immunity simultaneously, protect the body according to multiple mechanisms, and due to its own characteristics, it can be degraded soon in cytoplasm of transfected cell after immunization, thereby decreasing safety risk. In the response to the epidemic caused by the mutated coronavirus, mRNA vaccines have demonstrated unique advantages over other types of vaccines. Clinical trial data shows that the enhanced mRNA vaccine designed for variant strain has stronger neutralizing ability against mutated virus. Besides, the researching and developing period and manufacturing period of mRNA vaccine is shorter than that of the traditional vaccine, therefore, it is easy to achieve batch production with higher capacity of vaccine production.

Combination vaccine is made of two or more vaccine stock in specific ratio. It may prevent many kinds of diseases or diseases caused by different subtypes of one pathogenic microorganism, the former is called multiplex vaccine, and the latter is called multivalent vaccine. Combination vaccine is not equal to a simple superposition of any single vaccine, which not only does not aggravate the side effects after injection, but also effectively reduces the risk of adverse reactions that may occur due to multiple vaccinations.

Based on the current situation of different SARS-COV-2 variants are raging, combination immunization strategy provides new concept of preventing infection from different variants, decreasing vaccine injection times and reducing adverse immune response. Thus, the vaccine that may target to different SARS-COV-2 variants effectively and simultaneously is urgently needed.

### DESCRIPTION OF THE INVENTION

This invention provides an mRNA vaccine against SARS-COV-2, especially against SARS-COV-2 Delta variant (B.1.617.2), which can express prefusion stable recombinant S protein in vivo after being delivered to mouse, trigger body's cellular immunity and humoral immunity response, therefore inducing specific antibody in vivo. Compared with the 1^{st} generation of mRNA vaccine against SARS-Cov-2, the serum immunized by the vaccine of this invention has higher titer against SARS-COV-2 Delta variant S protein and stronger neutralizing ability against SARS-COV-2 Delta variant.

This invention also provides an mRNA vaccine against SARS-Cov-2, especially against SARS-COV-2 Omicron variant, which can express pre-fusion stable recombinant S protein in vivo after being delivered to mouse, trigger body's cellular immunity and humoral immunity response, therefore inducing specific antibody in vivo. Compared with the 15 generation of mRNA vaccine against SARS-COV-2 and the 2nd generation of mRNA vaccine against SARS-COV-2 Delta variant, the serum immunized by the vaccine of this invention has higher titer against SARS-COV-2 Omicron variant S protein and stronger neutralizing ability against SARS-COV-2 Omicron variant. Meanwhile, the vaccine of the application also has a certain inhibition effect on both wild type and Delta variant strains.

This invention also provides an mRNA vaccine composition against SARS-COV-2 and its variants (such as Delta and Omicron variants). The serum immunized by the mRNA vaccine composition of this invention can have inhibition effect on various SARS-COV-2 variants, with stronger neutralizing ability against wild type, Beta type, Gamma type, Alpha type, Delta type, Omicron type and Deltacron type SARS-COV-2.

SARS-COV-2, SARS-COV and MERS-COV belong to β-coronavirus of coronaviridae. The total length of SARS-COV-2 genome sequence is 29903 bp, with 79.5% identity with SARS-COV genome sequence and 40% identity with MERS-COV sequence. The main structure of SARS-COV-2 virus particles include single positive strand nucleic acid (ssRNA), spike protein(S), membrane protein (M), envelop protein (E) and nucleocapsid protein (N). Similar to other β-coronaviruses, the adsorption and invasion process of SARS-Cov-2 virus into the cells mainly relays on S protein; during this process, S protein assembles in the form of homotrimer, which has short cytoplasmic tail and a hydrophobic transmembrane domain to anchor the protein into the membrane.

S protein can be divided into receptor binding subunit S1 and membrane fusion subunit S2, S1 subunit can be divided into signal peptide (SP), N-terminal domain (NTD) and receptor binding domain (RBD), S2 subunit anchors on the membrane through transmembrane domain, which has basic elements required for the membrane fusion process, including: internal fusion peptide (FP), two heptad repeat (HR), transmembrane domain (TM), and cytoplasmic domain (CP) of C terminal.

The S protein consists of a signal peptide (SP) domain, an extracellular domain (ECD), a transmembrane (TM) domain and a cytoplasmic domain (CP) from N terminal to C terminal. The extracellular domain can be further divided into an N-terminal domain (NTD), a receptor binding domain (RBD), an intrinsic membrane fusion peptide domain (FP) and two heptad repeats (HR1 and HR2), belonging to Class I viral fusion protein. The signal peptide domain of S protein corresponds to the region of amino acid positions 1-13 of S protein; extracellular domain corresponds to the region of amino acid positions 14-1213 of S protein; transmembrane domain corresponds to the region of amino acid positions 1214-1237 of S protein; cytoplasmic domain corresponds to the region of amino acid positions 1238-1273 of S protein. The amino acid sequence of S protein is as shown in SEQ ID NO. 29. In this disclosure, unless otherwise defined, the amino acid positions of recombinant S protein are numbered according to the amino acid sequence of wild type S protein as shown in SEQ ID NO. 29.

After analyzing the pre-fusion structure of the S protein, it was found that the RBD domain of the S1 subunit undergoes a hinge-like conformational movement to hide or expose the key sites of receptor binding. Facing “down” means that the receptor is in a state of not being able to -bind, facing “up” means that the S protein is in a state of being able to -bind and is a relatively unstable state. This conformation allows the S protein to easily bind to the host receptor angiotensin converting enzyme 2 (ACE2). When RBD binds to the receptor, the S2 subunit transforms to the post-fusion conformation by inserting fusion peptide domain into the host cell membrane, HR1 and HR2 form an anti-parallel six-helix bundle (6HB), which form a fusion core together, and ultimately results in fusion of the viral membrane and cell membrane. With the cryo-electron microscopy, a large number of trimeric glycosylated S protein domains have been identified in the pre-fusion conformation. The pre-fusion S protein retains a large number of neutralizing antibody sensitive epitopes, while the post-fusion conformation allows the exposure of neutralizing sensitive epitopes only existing on pre-fusion conformation is minimized. Therefore, expressing pre-fusion stable form of SARS-COV-2 S trimeric protein is the key of developing safe and effective SARS-COV-2 vaccine. The optimized vaccine antigen retains the epitopes existing in pre-fusion confirmation of S protein, and induces antibody to inhibit virus fusion.

The term used herein “SARS-Cov-2 Delta variant”, “B.1.617.2”, “Delta type coronavirus” may be used interchangeably, and refers the SARS-Cov-2 subtype first appeared in India in October 2020, which mutates in various positions in genome compared with SARS-COV-2. These mutations trigger immune escape of coronavirus, resulting in that this virus has stronger adaptability to human body, faster spread speed, higher viral load, longer treatment period, easier to develope into severe disease and other characteristics, compared with other early coronavirus subtypes.

A report from Public Health England showed that the spread ability of Delta variant is 60% higher than that of Alpha variant (H Allen et. al., Increased household transmission of COVID-19 cases associated with SARS-COV-2 Variant of Concern B.1.617.2: a national case-control study 2021). The research showed that Delta variant develops resistance against the neutralizing antibody induced by vaccine (A Saito et. al., SARS-COV-2 spike P681R mutation enhances and accelerates viral fusion). The neutralizing antibody titer of 250 recipients vaccinated by Pfizer BNT162b2 against various VOC were detected, and the result showed that compared with wild type, the neutralizing antibody titer against Delta variant decrease 5.8 times, while that against Alpha variant only decrease 2.6 times (EC Wall et. al., Neutralising antibody activity against SARS-Cov-2 VOCs B.1.617.2 and B.1.351 by BNT162b2 vaccination. Lancet, 2021; 397 (10292): 2331-3).

The term used herein “SARS-COV-2 Omicron variant”, “B.1.1.529”, “Omicron type coronavirus” may be used interchangeably, and refers the SARS-COV-2 subtype first appeared in South Africa in November 2021, which mutates in various positions in the genome compared with wild type SARS-Cov-2, including mutations in S protein, ORF1a, ORF1b, ORF9b, M protein, E protein, N protein. These mutations result in not only stronger spread ability of the Omicron variant, but also enhanced resistance ability of this viral subtype against antibody protection effect, making it more resistant to the current SARS-COV-2 vaccine and escape from the immune response induced by vaccine.

In the first aspect, this invention provides a recombinant SARS-COV-2 spike protein (S protein), comprising following mutations in an extracellular domain, compared with a wild type S protein: T19R, G142D, E156G, F157 deletion, R158 deletion, A222V, L452R, T478K, D614G, P681R and D950N; wherein, the amino acid positions are numbered according to the amino acid sequence of the wild type S protein as shown in SEQ ID NO. 29.

In some embodiments, the S1/S2 cleavage site RRAR in extracellular domain of recombinant S protein (corresponding to amino acid position 682-685 of S protein) may be mutated to lose the ability of being cleaved by protease such as Furin-like protease and lysosomal protease. In some embodiments, the S1/S2 cleavage site RRAR of recombinant S protein may be mutated to GGSG.

In some embodiments, the S2 cleavage site KR in extracellular domain of recombinant S protein (corresponding to amino acid position 814-815 of S protein) may be mutated to lose the ability of being cleaved by protease such as Furin-like protease and lysosomal protease. In some embodiments, the S2 cleavage site KR of recombinant S protein or the antigenic fragment thereof may be mutated to AN.

During the intracellular packaging process of SARS-Cov-2 virus, S protein may be cleaved by protease such as Furin-like protease and lysosomal protease, and secretes the S protein with non-fusion state of S1 and S2 subunit. By mutating cleavage site of recombinant S protein such as S1/S2 cleavage site and/or S2 cleavage site, it may prevent the recombinant S protein from being cleaved by protease, therefore further improve its stability.

In some embodiments, the recombinant S protein also comprises K986P and V987P mutations. Introducing 2 proline mutations K986P and V987P in extracellular domain of the recombinant S protein may improve the stability of pre-fusion conformation.

In some embodiments, the recombinant S protein may not comprise functional fusion peptide domain (FP domain; corresponding to amino acid position 788-806 of S protein). For example, recombinant S protein may comprise mutated fusion peptide domain, such as by virtue of substitution, deletion, insertion and/or addition of one or more amino acid residues, causing the fusion peptide domain loses its natural function, such as the function of mediating the virus to fuse with the host cell membrane. Or, in some embodiments, recombinants S protein may not comprise fusion peptide domain.

By removing the functional fusion peptide domain from recombinant S protein, it may improve the stability of pre-fusion conformation, so that the pre-fusion conformation that retains and exposes S protein exists a large number of neutralizing antibody sensitive epitopes.

In some embodiments, the recombinant S protein may not comprise transmembrane domain (corresponding to the region of amino acid position 1214-1237 of S protein) and/or cytoplasmic domain (corresponding to the region of amino acid position 1238-1273 of S protein). In some embodiments, the recombinant S protein may not comprise a cytoplasmic domain. In some embodiment, the recombinant S protein may not comprise a transmembrane domain and a cytoplasmic domain. In some embodiments, the recombinant S protein may also comprise a trimer domain which, when being expressed, facilitates the recombinant S protein to form a trimer.

As used herein, “trimer domain” refers to the protein or peptide domain which forms a trimer spontaneously or under induction when being expressed. Many types of such trimer domains are known in this field. By including the trimer domain in the recombinant S protein (for example, by constructing a fusion protein), it is possible to promote the recombinant S protein to form a trimer conformation, and/or stabilize the trimer conformation of the recombinant S protein.

In some embodiments, the trimer domain of the recombinant S protein can comprise T4 phage fibritin trimer motif. In some embodiments, the T4 phage fibritin trimer motif can comprise the amino acid sequence as 18 shown in SEQ ID NO, (GYIPEAPRDGQAYVRKDGEWVLISTFL).

In some embodiments, the trimer domain can fuse with the recombinant S protein directly. In other embodiments, the trimer domain can fuse with the recombinant S protein by linker. In some embodiments, the trimer domain can fuse with the N terminal of the recombinant S protein. In other embodiments, the trimer domain can fuse with the C terminal of the recombinant S protein. For example, the trimer domain can fuse with the C terminal of the recombinant S protein by linker. In some embodiments, the linker sequence can comprise the sequence as shown in SEQ ID NO. 19 (SAIG).

In some embodiments, the recombinant S protein also comprises signal sequence; preferably, the signal sequence comprises immunoglobulin heavy chain variable region (IGHV) signal sequence. For example, the signal sequence can comprise the amino acid sequence as shown in SEQ ID NO. 17 (MDWIWRILFLVGAATGAHS).

In some embodiments, the recombinant S protein consists of from N terminal to C terminal, any one of the following items:
i) extracellular domain;
ii) extracellular domain, transmembrane domain and optionally cytoplasmic domain;
iii) extracellular domain and trimer domain;
iv) extracellular domain, transmembrane domain, optionally cytoplasmic domain, and trimer domain;
v) signal sequence, and extracellular domain;
vi) signal sequence, extracellular domain, transmembrane domain and optionally cytoplasmic domain;
vii) signal sequence, extracellular domain and trimer domain; and
viii) signal sequence, extracellular domain, transmembrane domain, optionally cytoplasmic domain, and trimer domain.

In some embodiments, compared with the wild type sequence, the extracellular domain comprises one or more following mutations:
1) T19R, G142D, E156G, F157 deletion, R158 deletion, A222V, L452R, T478K, D614G, P681R, and D950N;
2) S1/S2 cleavage site RRAR are mutated to lose the ability being cleaved by Furin-like proteases or lysosomal proteases, preferably, S1/S2 cleavage site is mutated to GGSG;
3) S2 cleavage sites KR are mutated to lose the ability being cleaved by Furin-like proteases or lysosomal proteases, preferably, S2 cleavage site is mutated to AN;
4) K986P and/or V987P mutation;
5) the fusion peptide domain is mutated to lose the function of mediating the fusion of virus with the host cell membrane; preferably fusion peptide domain deletion mutation.

In some embodiments, the signal sequence comprises immunoglobulin heavy chain variable region (IGHV) signal sequence. For example, the signal sequence has an amino acid sequence as shown in SEQ ID NO. 17.

In some embodiments, the trimer domain is T4 phage fibritin trimer motif. In some embodiments, the T4 phage fibritin trimer motif has the amino acid sequence as shown in SEQ ID NO. 18.

In preferred embodiments, the recombinant S protein consists of, from N terminal to C terminal, signal sequence, extracellular domain, transmembrane domain and cytoplasmic domain, compared with wild type sequence, the extracellular domain comprises the following mutations: T19R, G142D, E156G, F157 deletion, R158 deletion, A222V, L452R, T478K, D614G, P681R, D950N, S1/S2 cleavage sites RRAR is mutated to GGSG, and S2 cleavage sites KR is mutated to AN. For example, the recombinant S protein has the amino acid sequence as shown in SEQ ID NO. 1.

In another preferred embodiment, the recombinant S protein consists of, from N terminal to C terminal, signal sequence, extracellular domain, transmembrane domain and cytoplasmic domain, compared with wild type sequence, the extracellular domain comprises the following mutations: T19R, G142D, E156G, F157 deletion, R158 deletion, A222V, L452R, T478K, D614G, P681R, D950N, S1/S2 cleavage site RRAR is mutated to GGSG, and S2 cleavage site KR is mutated to AN. For example, the recombinant S protein has the amino acid sequence as shown in SEQ ID NO. 2.

In another preferred embodiment, the recombinant S protein consists of, from N terminal to C terminal, signal sequence, extracellular domain, transmembrane domain and cytoplasmic domain, compared with wild type sequence, the extracellular domain comprises the following mutations: T19R, G142D, E156G, F157 deletion, R158 deletion, A222V, 1,452R, T478K, D614G, P681R, D950N, K986P, V987P, S1/S2 cleavage site RRAR is mutated to GGSG, and S2 cleavage site KR is mutated to AN. For example, the recombinant S protein has the amino acid sequence as shown in SEQ ID NO. 3.

In another preferred embodiment, the recombinant S protein consists of, from N terminal to C terminal, signal sequence, extracellular domain and trimer domain, the signal sequence is immunoglobulin heavy chain variable region (IGHIV) signal sequence, preferably, the sequence as shown in SEQ ID NO. 17 (MDWIWRILFLVGAATGAHS); compared with wild type sequence, the extracellular domain comprises the following mutations: T19R, G142D, E156G, F157 deletion, R158 deletion, A222V, L452R, T478K, D614G, P681R, D950N, S1/S2 cleavage site RRAR is mutated to GGSG, S2 cleavage site KR is mutated to AN, and fusion peptide domain deletion mutation; and the trimer domain is T4 phage fibritin trimer motif, preferably, the sequence as shown in SEQ ID NO. 18 (GYIPEAPRDGQAYVRKDGEWVLLSTFL).

Preferably, the trimer domain fuses with C terminal of extracellular domain by linker. The linker sequence can be sequence as shown in SEQ ID NO. 19 (SAIG). For example, the recombinant S protein has the amino acid sequence as shown in SEQ ID NO. 4.

In another preferred embodiment, the recombinant S protein consists of, from N terminal to C′ terminal, signal sequence, extracellular domain and trimer domain, the signal sequence is immunoglobulin heavy chain variable region (IGHV) signal sequence, preferably, the sequence as shown in SEQ ID NO. 17 (MDWIWRILFLVGAATGAHS); compared with wild type sequence, the extracellular domain comprises the following mutations: T19R, G142D, E156G, F157 deletion, R158 deletion, A222V, L452R, T478K, D614G, P681R, D950N, K986P, V987P, S1/S2 cleavage site RRAR is mutated to GGSG, S2 cleavage site KR is mutated to AN, and fusion peptide domain deletion mutation; and the trimer domain is T4 phage fibritin trimer motif, preferably, the sequence as shown in SEQ ID NO. 18 (GYIPEAPRDGQAYVRKDGEWVLLSTEL). Preferably, the trimer domain fuses with C terminal of extracellular domain by linker. The linker sequence can be sequence as shown in SEQ ID NO. 19 (SAIG). For example, the recombinant S protein has the amino acid sequence as shown in SEQ ID NO. 5.

In some embodiments, the recombinant S protein has an amino acid sequence as shown in any one selected from SEQ ID NO. 1-5. In preferred embodiments, the recombinant S protein has an amino acid sequence as shown in any one selected from SEQ ID NO. 3-5.

In the second aspect, this invention provides mRNA encoding the recombinant S protein of the first aspect of this invention.

In some embodiments, mRNA from comprises cap structure, 5′-UTR, open reading flame (ORF) encoding recombinant S protein of this invention, 3′-UTR and polyA tailfrom 5′ to 3′.

In some embodiments, the cap structure is m⁷G5′ppp5′ (2′-OMe) NpG, wherein m⁷G is N7-methylguanosine, p is phosphoric acid, ppp is tri-phosphoric acid, 2′-OMe is 2′-methoxy modification; N is any nucleoside, such as adenosine (A), guanosine (G), cytosine (C) and uridine (L), or other naturally occurring nucleosides or modified nucleosides.

In some embodiments, the 5′-UTR may comprise a 5′-UTR derived from a gene selected from the following group or homologs, fragments or variants thereof: β-globin (HBB) gene, heat shock protein 70 (Hsp70) gene, axon Dynein heavy chain 2 (DNAH2) gene, 17β-hydroxysteroid dehydrogenase 4 (HSD17B4) gene. For example, the sequence of the variant can have at least 80%, at least 85%, at least 90%, at least 95%, at least 98% or at least 99% identity with wild type 5′-UTR sequence of corresponding gene.

In some embodiments, the 5′-UTR comprises a 5′-UTR derived from 17β-hydroxysteroid dehydrogenase 4 (HSD17B4) gene or homologs, fragments or variants thereof. In some embodiments, 5′-UTR comprises KOZAK sequence. In some embodiments, the 5′-UTR comprises a 5′-UTR derived from 17β-hydroxysteroid dehydrogenase 4 (HSD17B4) gene or homologs, fragments or variants thereof, and KOZAK sequence. In some embodiment, 5′-UTR comprises a sequence as shown in SEQ ID NO. 8 (GTCCCGCAGTOGGCGTCCAGCGGCTCTGCTTGTTCGTGTGTGTGTOGTTGCAGGCCT TATTC) and/or SEQ ID NO. 9 (AGATCTACCGGTGGTACCGCCACC).

In some embodiments, 3′-UTR comprises a 3′-UTR derived from a gene selected from the following group or homologs, fragments or variants thereof: albumin (ALB) gene, α-globin gene, β-globin (HIBB) gene, tyrosine hydroxylase gene, heat shock protein 70 (Hsp70) gene, lipoxygenase gene and collagen a gene. For example, the variant sequence can have at least 80%, at least 85%, at least 90%, at least 95%, at least 98% or at least 99% identity with wild type 3′-UTR sequence of corresponding gene. In some embodiments, 3′-UTR comprises a 3′-UTR derived from albumin (ALB) gene or homologs, fragments or variants thereof. Preferably, 3′-UTR comprises a sequence as shown in SEQ ID NO. 10 (AGCCAACACCCTGTCTAAAAAACATAAATTTCTTTAATCATTTTGCCTCTTTTCTCTG TGCTICAATTAATAAAAAATGGAAAGAACCT).

In some embodiments, the poly A tail can be 100-200 nucleotides, such as about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides. In some embodiments, the length of the poly A tail may be 100-150 nucleotides. In some embodiments, the length of the poly A tail can be about 120 nucleotides.

In some embodiments, the mRNA of this invention comprises a sequence as shown in any one of SEQ ID NO. 14-16, or consists of a sequence as shown in any one of SEQ ID NO. 14-16.

In some embodiments, one or more nucleotides of the mRNA may be modified. For example, one or more nucleotides of the mRNA (such as all nucleotides) each may be independently replaced by naturally occurring nucleotide analogues or artificially synthesized nucleotide analogues.

In some embodiments, the naturally occurring nucleotide analogues can be selected from pseudouridine, 2-thiouridine, 5-methyluridine, 5-methylcytidine and N6-methyladenosine. In some embodiments, the artificially synthesized nucleotide analogues can be selected from N1-methylpseudouridine and 5-ethynyluridine.

In some embodiments, one or more uridine triphosphate of the mRNA each may be independently replaced by pseudo-uridine triphosphate, 2-thio-uridine triphosphate, 5-methyl-uridine triphosphate, N1-methyl-pseudo-uridine triphosphate or 5-ethynyl-uridine triphosphate, and/or one or more cytidine triphosphate each may be independently replaced by 5-methyl-cytidine triphosphate, and/or one or more adenosine triphosphate (ATP) each may be independently replaced by N6-methyl-ATP.

In some embodiments, one or more uridine triphosphate of the mRNA each may be independently replaced by pseudo-uridine triphosphate, 1-methyl-pseudo-uridine triphosphate or 5-ethynyl-uridine triphosphate. In some embodiments, one or more cytidine triphosphate of the mRNA each may be independently replaced by 5-methyl-cytidine triphosphate.

In the third aspect, this invention provides recombinant SARS-COV-2 spike protein (S protein), comprising following mutations in an extracellular domain, compared with a wild type S protein: A67V, H69 deletion mutation, V70 deletion mutation, T95I, G142 deletion mutation, V143 deletion mutation, Y144 deletion mutation, Y145D, N211 deletion mutation, L212I, and insertion mutation of three amino acids E, P, E between R214 and D215 in NTD region; G339D, S371L, S373P, S375F, K417N, N440K, G446S, S477N, T478K, E484A, Q493R, G496S, Q498R, N501Y, Y505H in RBD region; T547K in SD1 region; D614G, H655Y, N679K, P681H in SD2 region; N764K, D796Y, N856K in the spacer region of SD2 and HR1; Q954H, N969K and L981F in HR1 region; wherein the positions of amino acid are numbered according to the wild type S protein amino acid sequence as shown in SEQ ID NO. 29.

In some embodiment, the S1/S2 cleavage site RRAR in extracellular domain of the recombinant S protein (corresponding to amino acid position 682-685 of S protein) may be mutated to lose the ability of being cleaved by protease such as Furin-like protease and lysosomal protease. In some embodiments, the S1/S2 cleavage site RRAR of recombinant S protein may be mutated to GGSG.

In some embodiments, the S2 cleavage site KR in extracellular domain of recombinant S protein (corresponding to amino acid position 814-815 of S protein) may be mutated to lose the ability of being cleaved by protease such as Furin-like protease and lysosomal protease. In some embodiments, the S2 cleavage site KR of recombinant S protein or the antigenic fragment thereof may be mutated to AN.

During the intracellular packaging process of SARS-COV-2 virus, S protein may be cleaved by protease such as Furin-like protease and lysosomal protease, and secrete the S protein with non-fusion state of S1 and S2 subunit. By mutating the cleavage site such as S1/S2 cleavage site and/or S2 cleavage site of recombinant S protein, it may avoid the recombinant S protein to be cleaved by protease, therefore further improve its stability.

In some embodiments, the recombinant S protein also comprises K986P and V987P mutations. Introducing 2 proline mutations K986P and V987P in extracellular domain of recombinant S protein can improve stability of pre-fusion conformation.

In some embodiments, the recombinant S protein may not comprise functional fusion peptide domain (FP domain; corresponding to amino acid position 788-806 of S protein). For example, the recombinant S protein may comprise mutated fusion peptide domain, such as by virture of substitution, deletion, insertion and/or addition of one or more amino acid residues, resulting in the loss of natural function of fusion peptide domain, such as the loss of the function of mediating the virus to fuse with the host cell membrane. Or, in some embodiments, the recombinants S protein may not comprise fusion peptide domain.

In some embodiments, the recombinant S protein may not comprise the transmembrane domain (corresponding to amino acid position 1214-1237 of S protein) and/or the cytoplasmic domain (corresponding to amino acid position 1238-1273 of S protein). In some embodiments, the recombinant S protein may not comprise the cytoplasmic domain. In some embodiment, the recombinant S protein may not comprise the transmembrane domain and the cytoplasmic domain. In some embodiments, the recombinant S protein may also comprise the trimer domain which facilitates the recombinant S protein to form the trimer when being expressed.

In some embodiments, the trimer domain of the recombinant S protein can comprise T4 phage fibritin trimer motif. In some embodiments, the T4 phage fibritin trimer motif can comprise the amino acid sequence as shown in SEQ ID NO. 18.

In some embodiments, the trimer domain can fuse with the recombinant S protein directly. In other embodiments, the trimer domain can fuse with the recombinant S protein by linker. In some embodiments, the trimer domain can fuse with the N terminal of the recombinant S protein. In other embodiments, the trimer domain can fuse with the C terminal of the recombinant S protein. For example, the trimer domain can fuse with the C terminal of recombinant S protein by linker. In some embodiments, the linker sequence can comprise the sequence as shown in SEQ ID NO. 19. In some embodiments, the recombinant S protein also comprises signal sequence; preferably, the signal sequence comprises immunoglobulin heavy chain variable region (IGHV) signal sequence. For example, the signal sequence can comprise the amino acid sequence as shown in SEQ ID NO. 17.

In some embodiments, the recombinant S protein consists of the following items from N terminal to C terminal: optionally signal sequence, extracellular domain, optionally transmembrane domain, optionally cytoplasmic domain and optionally trimer domain.

In some embodiments, the recombinant S protein consists of the following items from N terminal to C terminal: extracellular domain, optionally transmembrane domain and optionally cytoplasmic domain.

In preferred embodiments, the recombinant S protein consists of the following items from N terminal to C terminal: signal sequence, extracellular domain, transmembrane domain and cytoplasmic domain.

In some embodiments, compared with the wild type sequence, the extracellular domain comprises one or more of the following mutations:
1) A67V, H69 deletion mutation, V70 deletion mutation, T95I, G142 deletion mutation, V143 deletion mutation, Y144 deletion mutation, Y145D, N211 deletion mutation, L212I, and insertion mutation of three amino acids E, P, E between R214 and D215; G339D, S371L, S373P, S375F, K417N, N440K, G446S, S477N, T478K, F484A, Q493R, G496S, Q498R, N501Y, Y505H, T547K, D614G, H655Y, N679K, P681H, N764K, D796Y, N856K, Q954H, N969K and L981F;
2) S1/S2 cleavage site RRAR is mutated to lose the ability being cleaved by Furin-like proteases or lysosomal proteases, preferably, S1/S2 cleavage site is mutated to GGSG;
3) S2 cleavage site KR is mutated to lose the ability being cleaved by Furin-like proteases or lysosomal proteases, preferably, S2 cleavage site mutates is mutated to AN;
4) K986P and/or V987P mutation;

In some embodiments, the signal sequence comprises immunoglobulin heavy chain variable region (IGIIV) signal sequence. For example, the signal sequence can comprise the amino acid sequence as shown in SEQ ID NO. 17.

In some embodiments, the trimer domain of recombinant S protein is T4 phage fibritin trimer motif. In some embodiments, the T4 phage fibritin trimer motif has the amino acid sequence as shown in SEQ ID NO. 18.

In preferred embodiments, the recombinant S protein consists of, from N terminal to C terminal, signal sequence, extracellular domain, transmembrane domain and cytoplasmic domain, compared with the wild type sequence, the extracellular domain has the following mutations: A67V, H169 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371L, S373P, S375F, K417N, N440K, G446S, S477N, T478K, F484A, Q493R, G496S, Q498R, N501Y, Y505H, T547K, D614G, H655Y, N679K, P681H, N764K, D796Y, N856K, Q954H, N969K and L981F. For example, the recombinant S protein has the amino acid sequence as shown in SEQ ID NO, 20.

In other preferred embodiments, the recombinant S protein consists of, from N terminal to C′ terminal, signal sequence, extracellular domain, transmembrane domain and cytoplasmic domain, compared with the wild type sequence, the extracellular domain has the following mutations: A67V, H69 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K and L981F; S1/S2 cleavage site is mutated to GGSG; and S2 cleavage site is mutated to AN. For example, the recombinant S protein has the amino acid sequence as shown in SEQ ID NO. 21.

In other preferred embodiments, the recombinant S protein consists of, from N terminal to C terminal, signal sequence, extracellular domain, transmembrane domain and cytoplasmic domain, compared with the wild type sequence, the extracellular domain has the following mutations: A67V, H69 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371I, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K and L981F; K986P and V987P. For example, the recombinant S protein has the amino acid sequence as shown in SEQ ID NO. 22.

In other preferred embodiments, the recombinant S protein consists of, from N terminal to C terminal, signal sequence, extracellular domain, transmembrane domain and cytoplasmic domain, compared with the wild type sequence, the extracellular domain has the following mutations: A67V, H69 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371L, S373P, S375F, K417N, N440K, G446S, S477N, T478K, F484A, Q493R, G496S, Q498R, N501Y, Y505H, T547K, D614G, H655Y, N679K, P681H, N764K, D796Y, N856K, Q954H, N969K and L981F; K986P and V987P; S1/S2 cleavage site is mutated to GGSG. For example, the recombinant S protein has the amino acid sequence as shown in SEQ ID NO. 23.

In other preferred embodiments, the recombinant S protein consists of, from N terminal to C terminal, signal sequence, extracellular domain, transmembrane domain and cytoplasmic domain, compared with the wild type sequence, the extracellular domain has the following mutations: A67V, H69 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K and L981F; K986P and V987P; S2 cleavage site KR is mutated to AN. For example, the recombinant S protein has the amino acid sequence as shown in SEQ ID NO. 24.

In other preferred embodiments, the recombinant S protein consists of, from N terminal to C′ terminal, signal sequence, extracellular domain, transmembrane domain and cytoplasmic domain, compared with the wild type sequence, the extracellular domain has the following mutations: A67V, H69 deletion mutation, V70 deletion mutation, T95I, G142 deletion mutation, V143 deletion mutation, Y144 deletion mutation, Y145D, N211 deletion mutation, L212I, insertion mutation of three amino acids E, P, E between R214 and D215, G339D, S371L, S373P, S375F, K417N, N440K, G446S, S477N, T478K, F484A, Q493R, G496S, Q498R, N501Y, Y505H, T547K, D614G, H655Y, N679K, P681H, N764K, D796Y, N856K, Q954H, N969K and L981F; K986P and V987P; S1/S2 cleavage site RRAR is mutated to GGSG; and S2 cleavage site KR is mutated to AN. For example, the recombinant S protein has the amino acid sequence as shown in SEQ ID NO. 25.

In some embodiments, the recombinant S protein has an amino acid sequence as shown in any one selected from SEQ ID NO. 20-25. In preferred embodiments, the recombinant S protein has an amino acid sequence as shown in any one selected from SEQ ID NO. 23-25. In the most preferred embodiment, the recombinant S protein has the amino acid sequence as shown in SEQ ID NO. 25.

In the fourth aspect, this invention provides mRNA which encodes the recombinant S protein in the third aspect of this invention.

In some embodiments, the mRNA comprises cap structure, 5′-UTR, open reading flame (ORF) encoding recombinant S protein of this invention, 3′-UTR and polyA tail from 5′ to 3′.

In some embodiments, the cap structure may have m⁷G5′ppp5′ (2′-OMe)NpG, wherein m⁷G is N7-methylguanosine, p is phosphoric acid, ppp is triphosphoric acid, 2′-OMe is 2′-methoxy modification; N is any nucleoside, such as adenosine (A), guanosine (G), cytosine (C) and uridine (U), or other naturally occurring nucleosides or modified nucleosides.

In some embodiments, the 5′-UTR may comprise a 5′-UTR derived from the gene selected from the following group or homologs, fragments or variants thereof: β-globin (HBB) gene, heat shock protein 70 (Hsp70) gene, axon Dynein heavy chain 2 (DNAH2) gene, 17β-hydroxysteroid dehydrogenase 4 (HSD17B4) gene. For example, the variant sequence can have at least 80%, at least 85%, at least 90%, at least 95%, at least 98% or at least 99% identity with wild type 5′-UTR sequence of corresponding gene.

In some embodiments, the 5′-UTR comprises a 5′-UTR derived from 17β-hydroxysteroid dehydrogenase 4 (HSD17B4) gene or homologs, fragments or variants thereof. In some embodiments, the 5′-UTR comprises KOZAK sequence. In some embodiments, the 5′-UTR comprise a 5′-UTR derived from 17β-hydroxysteroid dehydrogenase 4 (HSD17B4) gene or homologs, fragments or variants thereof, and KOZAK sequence. In some embodiment, the 5′-UTR comprises sequence as shown in SEQ ID NO. 8 and/or SEQ ID NO. 9.

In some embodiments, the 3′-UTR comprises a 3′-UTR derived from the gene selected from the following group or homologs, fragments or variants thereof: albumin (ALB) gene, α-globin gene, β-globin (HBB) gene, tyrosine hydroxylase gene, heat shock protein 70 (Hsp70) gene, lipoxygenase gene and collagen a gene. For example, the variant sequence can have at least 80%, at least 85%, at least 90%, at least 95%, at least 98% or at least 99% identity with wild type 3′-UTR sequence of corresponding gene. In some embodiments, the 3′-UTR comprises a 3′-UTR derived from albumin (ALB) gene or homologs, fragments or variants thereof. Preferably, the 3′-UTR comprises sequence as shown in SEQ ID NO. 10.

In some embodiments, the polyA tail can be 100-200 nucleotides, such as about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides. In some embodiments, the length of the polyA tail can be about 100-150 nucleotides. In some embodiments, the length of the polyA tail can be about 120 nucleotides.

In some embodiments, the mRNA of this invention comprises sequence as shown in SEQ ID NO. 27, or consists of sequence as shown in SEQ ID NO. 27.

In some embodiments, one or more nucleotides of the mRNA may be modified. For example, one or more nucleotides of the mRNA (such as all nucleotides) each may be independently replaced by naturally occurring nucleotide analogues or artificially synthesized nucleotide analogues.

In some embodiments, the naturally occurring nucleotide analogues can be selected from pseudouridine, 2-thiouridine, 5-methyluridine, 5-methylcytidine and N6-methyladenosine. In some embodiments, the artificially synthesized nucleotide analogues can be selected from N1-methylpseudouridine and 5-ethynyluridine.

In some embodiments, one or more uridine triphosphate of the mRNA each may be independently replaced by pseudo-uridine triphosphate, 2-thio-uridine triphosphate, 5-methyl-uridine triphosphate, V1-methyl-pseudo-uridine triphosphate or 5-ethynyl-uridine triphosphate, and/or one or more cytidine triphosphate each may be independently replaced by 5-methyl-cytidine triphosphate, and/or one or more ATP each may be independently replaced by N6-methyl-ATP.

In some embodiments, one or more uridine triphosphate of the mRNA each may be independently replaced by pseudo-uridine triphosphate, 1-methyl-pseudo-uridine triphosphate or 5-ethynyl-uridine triphosphate. In some embodiments, one or more cytidine triphosphate of the mRNA each may be independently replaced by 5-methyl-cytidine triphosphate.

In the fifth aspect, this invention provides composition which comprises the recombinant S protein in the first aspect of this invention or the mRNA in the second aspect of this invention, and the recombinant S protein in the third aspect of this invention or the mRNA in the fourth aspect of this invention.

In some embodiments of the composition of this invention, the composition comprises the recombinant S protein in the first aspect or the recombinant S protein in the third aspect of this invention. In some embodiments, the composition comprises the recombinant S protein in the first aspect and the mRNA in the fourth aspect of this invention. In some embodiments, the composition comprises mRNA in the second aspect and the recombinant S protein in the third aspect of this invention. In some embodiments, the composition comprises the mRNA in the second aspect and the mRNA in the fourth aspect of this invention.

In some embodiments, the composition comprises mRNA having an amino acid sequence as shown in any one of SEQ ID NO. 14-16 and SEQ ID NO. 27. In preferred embodiments, the composition comprises mRNA having an amino acid sequence as shown in any one of SEQ ID NO. 14 and SEQ ID NO. 27.

In some embodiments, the molar ratio between the 2 types of recombinant S proteins or between the 2 types of mRNA in the composition is 1-3:1-3, such as 1:1, 1:1.5, 1:2, 1:2.5, 1:3, 1.5:2.5, 2:1.5, 2:2.5, 2:3, 2.5:3, preferably 1:1. In preferred embodiments, the molar ratio of the recombinant S protein in the first aspect to the recombinant S protein in the third aspect of this invention is 1-3:1-3, such as 1:1, 1:1.5, 1:2, 1:2.5, 1:3, 1.5:1, 1.5:2, 1.5:2.5, 2:1, 2:1.5, 2:2.5, 2:3, 2.5:1, 2.5:1.5, 2.5:2, 2.5:3, 3:1, 3:2, 3:2.5, preferably, 1:1. In preferred embodiments, the molar ratio of the mRNA in the second aspect to the mRNA in the fourth aspect of this invention is 1-3:1-3, such as 1:1, 1:1.5, 1:2, 1:2.5, 1:3, 1.5:1, 1.5:2, 1.5:2.5, 2:1, 2:1.5, 2:2.5, 2:3, 2.5:1, 2.5:1.5, 2.5:2, 2.5:3, 3:1, 3:2, 3:2.5, preferably, 1:1.

In some embodiments, the composition also comprises the following recombinant S protein or mRNA encoding the same:
(a) a recombinant S protein comprising following mutations compared with a wild type S protein: K986P and V987P; and/or
(b) a recombinant S protein comprising following mutations compared with a wild type S protein: G75V, T76I, R246 deletion, S247 deletion, Y248 deletion, L249 deletion, T250 deletion, P251 deletion, G252 deletion, D253N, 1452Q, F490S, D614G, T859N; K986P; and V987P; and/or
(c) a recombinant S protein comprising following mutation compared with a wild type S protein: mutation of a S1/S2 cleavage site to GGSG; K986P; and V987P; and/or
(d) a recombinant S protein comprising following mutations compared with a wild type S protein: mutation of a S2 cleavage site to AN; K986P; and V987P;
and/or
(e) a recombinant S protein comprising following mutations compared with a wild type S protein: mutation of a S1/S2 cleavage site to GGSG; mutation of a S2 cleavage site to AN; K986P; and V987P;
and/or
(f) a recombinant S protein comprising following mutations compared with a wild type S protein: G75V, T76I, R246 deletion, S247 deletion, Y248 deletion, L249 deletion, T250 deletion, P251 deletion, G252 deletion, D253N, L452Q, F490S, D614G, T859N; mutation of a S1/S2 cleavage site to GGSG; K986P; and V987P;
and/or
(g) a recombinant S protein comprising following mutations compared with a wild type S protein: G75V, T76I, R246 deletion, S247 deletion, Y248 deletion, L249 deletion, T250 deletion, P251 deletion, G252 deletion, D253N, 1452Q, F490S, D614G, T859N; mutation of a S2 cleavage site to AN; K986P; and V987P;
and/or
(h) a recombinant S protein comprising following mutations compared with a wild type S protein: G75V, T76I, R246 deletion, S247 deletion, Y248 deletion, L249 deletion, T250 deletion, P251 deletion, G252 deletion, D253N, 1452Q, F490S, D614G, T859N; mutation of a $1/$2 cleavage site to GGSG; mutation of a S2 cleavage site to AN; K986P; and V987P.

In some embodiments, the S1/S2 cleavage site RRAR of the recombinant S protein (a) may be mutated to lose the ability of being cleaved by Furin-like protease and lysosomal protease; preferably, the S1/S2 cleavage site RRAR is mutated to GGSG.

In some embodiments, the S2 cleavage site KR of the recombinant S protein (a) may be mutated to lose the ability of being cleaved by Furin-like protease and lysosomal protease; preferably, the S2 cleavage site KR is mutated to AN.

In some embodiments, the recombinant S protein (a) further comprises trimer domain, the trimer domain when being expressed accelerates the recombinant S protein (a) to form a trimer. In some embodiments, the trimer domain of the recombinant S protein (a) can comprise T4 phage fibritin trimer motif. In some embodiments, the T4 phage fibritin trimer motif may have the amino acid sequence as shown in SEQ ID NO. 18.

In some embodiments, trimer domain of recombinant S protein (a) can directly fuse with the recombinant S protein (a). In other embodiments, the trimer domain can fuse with the recombinant S protein (a) by linker. In some embodiments, the trimer domain can fuse with N terminal of the recombinant S protein (a). In other embodiments, the trimer domain can fuse with C terminal of the recombinant S protein (a). For example, the trimer domain can fuse with C terminal of the recombinant S protein (a) by linker. In some embodiments, the linker sequence can comprise sequence as shown in SEQ ID NO. 19.

In some embodiments, the recombinant S protein (a) may not comprise functional fusion peptide domain (FP domain). For example, the recombinant S protein (a) can comprise mutated fusion peptide domain, for example, by virtue of substitution, deletion, insertion and/or addition of one or more amino acid residues, resulting in the loss of natural function of fusion peptide domain, for example, the function of mediating the fusion of virus with the host cell membrane. Or, in some embodiments, recombinant S protein (a) may not comprise the fusion peptide domain.

In some embodiments, the recombinant S protein (a) may not comprise transmembrane domain and/or cytoplasmic domain. In some embodiments, the recombinant S protein (a) may not comprise cytoplasmic domain. In some embodiments, the recombinant S protein (a) may not comprise transmembrane domain and cytoplasmic domain.

In some embodiments, the recombinant S protein (a) further comprises signal sequence; preferably, the signal sequence comprises immunoglobulin heavy chain variable region (IGHV) signal sequence. For example, the signal sequence can comprise the amino acid sequence as shown in SEQ ID NO. 17.

In some embodiments, the recombinant S protein (a) consists of, from N terminal to C terminal, any one of the following item:
i) extracellular domain and trimer domain;
ii) extracellular domain, transmembrane domain and trimer domain;
iii) signal sequence, extracellular domain and trimer domain; and
iv) signal sequence, extracellular domain, transmembrane domain, and trimer domain.

In some embodiments, compared with the wild type sequence, the extracellular domain comprises one or more of following mutations:
1) the S1/S2 cleavage site RRAR is mutated to lose the ability being cleaved by Furin-like proteases or lysosomal proteases, preferably, the S1/S2 cleavage site is mutated to GGSG;
2) the S2 cleavage site KR is mutated to lose the ability being cleaved by Furin-like proteases or lysosomal proteases, preferably, the S2 cleavage site is mutated to AN;
3) K986P and/or V987P mutation;
4) the fusion peptide domain is mutated to lose the function of mediating the fusion of virus with the host cell membrane; preferably fusion peptide domain deletion mutation.

In preferred embodiments, recombinant S protein (a) consists of any one of the following item from N terminal to C terminal:
i) extracellular domain and trimer domain;
ii) extracellular domain, transmembrane domain and trimer domain;
iii) signal sequence, extracellular domain and trimer domain; and
iv) signal sequence, extracellular domain, transmembrane domain, and trimer domain;
wherein the amino acid sequence of the extracellular domain is the sequence corresponding to amino acid position 14-1213 of the amino acid sequence as shown in SEQ ID NO. 29 and the sequence is obtained by the following mutations: K986P and V987P substitution at amino acid positions 986 and 987 and no other mutations at amino acid positions 817-987 in the amino acid sequence as shown in SEQ ID NO. 29, and the S2 cleavage site KR in the extracellular domain is mutated to lose the ability of being cleaved by Furin-like protease and lysosomal protease, and the S1/S2 cleavage site RRAR in the extracellular domain is mutated to lose the ability of being cleaved by Furin-like protease and lysosomal protease, and
wherein the trimer domain when being expressed accelerates the recombinant S protein (a) to form a trimer, wherein the trimer domain is T4 phage fibritin trimer motif, and the trimer domain fuses with C terminal of the extracellular domain or transmembrane domain by optional linker sequence.

In preferred embodiments, the recombinant S protein (a) has an amino acid sequence as shown in any one selected from SEQ ID NO. 30-33. In preferred embodiments, the mRNA encoding recombinant S protein (a) has an amino acid sequence as shown in any one selected from SEQ ID NO. 34-37.

The structure of the recombinant S protein (a) and the mRNA encoding the same may refer to Chinese patent application No, 202011369776.2, which is herein incorporated by reference in its entirety.

In other embodiments, the S1/S2 cleavage site RRAR of the recombinant protein (b) is mutated to lose the ability of being cleaved by protease such as Furin-like protease and lysosomal protease; preferably, the S1/S2 cleavage site RRAR is mutated to GGSG.

In some embodiments, the S2 cleavage site KR of the recombinant protein (b) is mutated to lose the ability of being cleaved by protease such as Furin-like protease and lysosomal protease; preferably, the S2 cleavage site KR is mutated to AN.

In some embodiments, the recombinant S protein (b) may not include transmembrane domain and/or cytoplasmic domain. In some embodiments, the recombinant S protein (b) may not include cytoplasmic domain. In some embodiments, the recombinant S protein (b) may not include transmembrane domain and cytoplasmic domain.

In preferred embodiments, the recombinant S protein (b) consists of any one of the following items from N terminal to C terminal:
i) extracellular domain, optionally transmembrane domain and optionally cytoplasmic domain;
ii) signal sequence, extracellular domain, optionally transmembrane domain and optionally cytoplasmic domain:

In some embodiments, compared with the wild type sequence, the extracellular domain has one or more of the following mutations:
1) G75V, T76I, R246 deletion, S247 deletion, Y248 deletion, L249 deletion, T250 deletion, P251 deletion, G252 deletion, D253N, L452Q, F490S, D614G, T859N;
2) the S1/S2 cleavage site RRAR is mutated to lose the ability being cleaved by Furin-like proteases or lysosomal proteases, preferably, the S1/S2 cleavage site is mutated to GGSG;
3) the S2 cleavage site KR is mutated to lose the ability being cleaved by Furin-like proteases or lysosomal proteases, preferably, the S2 cleavage site is mutated to AN;
4) K986P and/or V987P.

In preferred embodiments, recombinant S protein (b) consists of, from N terminal to C terminal, signal sequence, extracellular domain, transmembrane domain and cytoplasmic domain, compared with wild type sequence, the extracellular domain has the following mutations: G75V, T76I, R246 deletion, S247 deletion, Y248 deletion, L249 deletion, T250 deletion, P251 deletion, G252 deletion, D253N, 1452Q, F490S, D614G, and T859N.

In other preferred embodiments, the recombinant S protein (b) consists of, from N terminal to C terminal, signal sequence, extracellular domain, transmembrane domain and cytoplasmic domain, compared with wild type sequence, the extracellular domain comprises the following mutations: G75V, T76I, R246 deletion, S247 deletion, Y248 deletion, L249 deletion, T250 deletion, P251 deletion, G252 deletion, D253N, L452Q, F490S, D614G, T859N, the S1/S2 cleavage site RRAR is mutated to GGSG and the S2 cleavage site KR is mutated to AN. For example, the recombinant S protein (b) has an amino acid sequence as shown in SEQ ID NO. 38.

In other preferred embodiments, the recombinant S protein (b) consists of, from N terminal to C terminal, signal sequence, extracellular domain, transmembrane domain and cytoplasmic domain, compared with wild type sequence, the extracellular domain comprises the following mutations: G75V, T76I, R246 deletion, S247 deletion, Y248 deletion, L249 deletion, T250 deletion, P251 deletion, G252 deletion, D253N, 1452Q, F490S, D614G, T859N, K986P and V987P. For example, the recombinant S protein (b) has an amino acid sequence as shown in SEQ ID NO. 39.

In other preferred embodiments, the recombinant S protein (b) consists of, from N terminal to C terminal, signal sequence, extracellular domain, transmembrane domain and cytoplasmic domain, compared with wild type sequence, the extracellular domain comprises the following mutations: G75V, T76I, R246 deletion, S247 deletion, Y248 deletion, L249 deletion, T250 deletion, P251 deletion, G252 deletion, D253N, 1452Q, F490S, D614G, T859N, K986P, V987P and the S1/S2 cleavage site RRAR is mutated to GGSG. For example, the recombinant S protein (b) has an amino acid sequence as shown in SEQ ID NO. 40.

In other preferred embodiments, the recombinant S protein (b) consists of, from N terminal to C terminal, signal sequence, extracellular domain, transmembrane domain and cytoplasmic domain, compared with wild type sequence, the extracellular domain comprises the following mutations: G75V, T76I, R246 deletion, S247 deletion, Y248 deletion, L249 deletion, T250 deletion, P251 deletion, G252 deletion, D253N, L452Q, F490S, D614G, T859N, K986P, V987P and the S2 cleavage site KR is mutated to AN. For example, the recombinant S protein (b) has an amino acid sequence as shown in SEQ ID NO. 41.

In other preferred embodiments, the recombinant S protein (b) consists of, from N terminal to C terminal, signal sequence, extracellular domain, transmembrane domain and cytoplasmic domain, compared with wild type sequence, the extracellular domain comprises the following mutations: G75V, T76I, R246 deletion, S247 deletion, Y248 deletion, L249 deletion, T250 deletion, P251 deletion, G252 deletion, D253N, L452Q, F490S, D614G, T859N, K986P, V987P, the S1/S2 cleavage site RRAR is mutated to GGSG and the S2 cleavage site KR is mutated to AN. For example, the recombinant S protein (b) has an amino acid sequence as shown in SEQ ID NO. 42.

In preferred embodiments, recombinant S protein (b) has an amino acid sequence as shown in any one selected from SEQ ID NO. 38-42. In preferred embodiments, the mRAN encoding recombinant S protein (b) has an sequence as shown in SEQ ID NO. 43.

The structure of the recombinant S protein (b) and the mRNA encoding the same may refer to Chinese patent application No, 202210159238.3, which is herein incorporated by reference in its entirety.

In some embodiments, the composition of this invention further comprises one or more pharmaceutically acceptable carrier, excipient or diluent.

As used herein, “pharmaceutically acceptable” refers to those carriers, excipients or diluents which are, within the scope of sound medical judgment, suitable for use in contact with human and animal tissues without undue toxicity, irritation, allergic response or other problems or complications, and are commensurate with a reasonable benefit/risk ratio.

Exemplary carriers for use in the composition of this invention include saline, buffered saline, dextrose and water. The exemplary excipient for use in the composition of this invention includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. It is within the knowledge of the skilled person to select suitable excipients for preparing the composition of this invention. Typically, choice of suitable excipients will inter alia depend on the active agent used, the disease to be treated, and the desired formulation of the composition.

The composition of this invention can be formulated in various forms, depending on the active agent (such as mRNA) used, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. Processes known per se for producing medicaments are indicated in 22nd edition of Remington's Pharmaceutical Sciences (Ed, Maack Publishing Co, Easton, Pa., 2012) and may include, for instance conventional mixing, dissolving, granulating, dragee-making, levigating, emulsifying, encapsulating, entrapping or lyophilizing processes.

In some embodiments, the composition can be vaccine composition, optionally, vaccine composition further comprises one or more adjuvants.

As used herein, the term “vaccine composition” refers to a biological preparation which induces or improves immunity to a specific disease. Challenging an individual's immune system with vaccine composition induces the formation and/or propagation of immune cells which specifically recognize the compound comprised by the vaccine. At least a part of said immune cells remains viable for a period of time which can extend to 10, 20 or 30 years after vaccination. If the individual's immune system encounters the pathogen from which the compound capable of eliciting an immune response was derived within the aforementioned period of time, the immune cells generated by vaccination are reactivated and enhance the immune response against the pathogen as compared to the immune response of an individual which has not been challenged with the vaccine and encounters immunogenic compounds of the pathogen for the first time.

As used herein, “vaccinating”, “inoculating”, “immunization” or “vaccination” refers to the administration of a vaccine to a subject, with the aim to prevent the subject from developing one or more symptoms of a disease. In principle, the vaccination comprises an prime vaccination and optionally one or more boost vaccinations. The prime vaccination or the prime immunization is defined as the initial administration schedule for administering the composition or unit dose as disclosed herein to establish a protective immune response. The boost vaccination or boost immunization refers to an administration or administration schedule which takes place after the prime vaccination e.g. at least 1 week, 2 weeks, 1 month, 6 months, 1 year or even 5 or 10 years after the last administration of the prime vaccination schedule. The boost administration attempts at enhancing or reestablishing the immune response of the prime vaccination.

An immune response to a composition or vaccine composition of this invention is the development in a subject of a humoral and/or a cellular immune response to an antigenic protein existed in the composition. For purposes of this invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s), CTLs have specificity for peptide antigens that are presented in association with proteins encoded by the major histocompatibility complex (MHC) and expressed on the surfaces of cells, CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4, and CD8+ T-cells.

Thus, an immune response may be one that stimulates CTIs, and/or the production or activation of helper T-cells. The production of chemokines and/or cytokines may also be stimulated. The composition or vaccine composition of this invention may also elicit an antibody-mediated immune response. Hence, an immune response may include one or more of the following effects: the production of antibodies (e.g., IgA or IgG) by B-cells; and/or the activation of suppressor, cytotoxic, or helper T-cells and/or T-cells directed specifically to a protein existed in the vaccine. These responses may serve to neutralize infectivity, and/or mediate antibody-complement, or antibody dependent cell cytotoxicity (ADCC) to provide protection to an immunized individual. Such responses can be determined using standard immunoassays and neutralization assays, well known in the art.

As used herein, the term “adjuvant” refers to agents that augment, stimulate, activate, potentiate, or modulate the immune response to the active ingredient of the composition at either the cellular or humoral level, e.g. immunologic adjuvants stimulate the response of the immune system to the actual antigen, but have no immunological effect themselves. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ), particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g polyarginine or polylysine). Preferably, adjuvants are selected from aluminum adjuvant (e.g. aluminum hydroxide, aluminum phosphate, aluminum sulfate, alum), MF59, AS03, virion (e.g. hepatitis virus virions and influenza virus virions), AS04, thermally reversible oil-in-water emulsion, ISA51, Freund's adjuvant, IL-12, CpG motif, manose or any combination thereof.

In some embodiments, the composition or vaccine composition further comprises one or more other therapeutic agents. For example, the therapeutic agents can be selected from other antigenic proteins or polypeptides, antibodies, hormones or hormone analogs, and small molecule drugs.

In the sixth aspect, this invention provides DNA which encodes the mRNA in the second aspect and/or the mRNA in the fourth aspect of this invention. In some embodiments, the DNA of this invention encodes the mRNA in the second aspect of this invention. In some embodiments, the DNA of this invention encodes the mRNA in the fourth aspect of this invention. In some embodiments, this invention provides the DNA which encodes the mRNA in the second aspect and the mRNA in the fourth aspect of this invention. In some embodiments, the DNA of this invention can be used in preparing the mRNA of this invention by transcription in vitro.

In some embodiments, the DNA of this invention comprises a sequences as shown in any one of SEQ ID NO. 11-13 and 26, or consists of a sequences as shown in any one of SEQ ID NO, 11-13 and 26.

In the seventh aspect, this invention provides recombinant plasmid which comprises the DNA in the sixth aspect of this invention.

In some embodiments, the recombinant plasmid is a pT7TS plasmid.

In some embodiments, the recombinant plasmid further comprises a original sequence (Ori), a T7 promoter, 5′-UTR and 3′-UTR.

In some embodiments, the Ori is ColE1 type Ori, Preferably, the Ori comprises the sequence as shown in SEQ ID NO. 6, or consists of the sequence as shown in SEQ ID NO. 6.

In some embodiments, the T7 promoter comprises the sequence as shown in SEQ ID NO. 7 (TAATACGACTCACTATAATG), or consists of the sequence as shown in SEQ ID NO. 7.

In some embodiments, the 5′-UTR can comprise a 5′-UTR derived from the gene selected from the following group or homologs, fragments or variants thereof: β-globin (HBB) gene, heat shock protein 70 (Hsp70) gene, axon Dynein heavy chain 2 (DNAH2) gene, 17β-hydroxysteroid dehydrogenase 4 (HSD17B4) gene. For example, the variant sequence can have at least 80%, at least 85%, at least 90%, at least 95%, at least 98% or at least 99% identity with wild type 5′-UTR sequence of corresponding gene.

In some embodiments, the 5′-UTR comprises the 5′-UTR derived from HSD17B4 gene or homologs, fragments or variants thereof. In some embodiments, the 5′-UTR comprises KOZAK sequence. In some embodiments, the 5′-UTR comprises the 5′-UTR derived from HSD17B4 gene or homologs, fragments or variants thereof, and KOZAK sequence. In some embodiments, the 5′-UTR comprises sequences as shown in SEQ ID NO. 8 and/or SEQ ID NO. 9.

In some embodiments, the 3′-UTR may comprise a 3′-UTR of a gene selected from the following group or homologs, fragments or variants thereof: albumin (ALB) gene, α-globin gene, β-globin (HBB) gene, tyrosine hydroxylase gene, heat shock protein 70 (Hsp70) gene, lipoxygenase gene and collagen a gene. For example, the variant sequence can have at least 80%, at least 85%, at least 90%, at least 95%, at least 98% or at least 99% identity with wild type 3′-UTR sequence of corresponding gene.

In some embodiments, the 3′-UTR comprises the 3′-UTR derived from ALB gene or homologs, fragments or variants thereof. Preferably, the 3′-UTR comprises sequence as shown in SEQ ID NO. 10.

In some embodiments, the recombinant plasmid further comprises polyA, resistance gene promoter and resistance gene.

In some embodiments, the length of polyA tail can be 100-200 nucleotides, such as about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides. In some embodiments, the length of polyA tail may be 100-150 nucleotides. In some embodiments, the length of the polyA tail can be about 120 nucleotides.

In some embodiments, the resistance gene promoter is ampicillin resistance gene promoter.

In some embodiments, the resistance gene is kanamycin sulfate resistance gene.

In preferred embodiments, the recombinant plasmid comprises nucleic acid sequence as shown in SEQ ID NO. 28, or consists of nucleic acid sequence as shown in SEQ ID NO. 28.

In the eighth aspect, this invention provides mRNA-carrier particle which comprises the mRNA in the second aspect and/or the mRNA in the fourth aspect of this invention, and carrier material encapsulating the mRNA.

In some embodiments, the carrier material can be selected from protamine, lipid nanoparticles (LNP), polymer materials and inorganic nanoparticles. In preferred embodiments, the carrier material is LNP.

In some embodiments, the LNP can comprise one or more of ionic lipid, pegylated lipids, cholesterol and derivatives thereof and phospholipid. For example, the LNP can comprise any one, any two, any three or all four of ionic lipid, pegylated lipids, cholesterol and derivatives thereof and phospholipid.

In the ninth aspect, this invention provides a method for preventing and/or treating a disease or condition associated with SARS-COV-2 infection in a subject, which comprises administering to a subject an effective amount of the recombinant S protein, mRNA, the composition, the recombinant plasmid, or mRNA-carrier particle of the invention.

The term “preventing” or “prevention” or “treating” or “treatment” used herein refers to a reduction in risk of acquiring or developing a disease or disorder (i.e., causing at least one of the clinical symptoms of the disease not to develop in a subject not yet exposed to a disease-causing agent, or predisposed to the disease in advance of disease onset). For example, treating can comprise: (i) preventing a disease, disorder and/or symptom from occurring in a patient that may be predisposed to the disease, disorder, and/or symptom but has not yet been diagnosed as having it; (ii) inhibiting the disease, disorder, and/or symptom, i.e., arresting its development; and (iii) relieving the disease, disorder, and or symptom, i.e., causing regression of the disease, disorder, and/or symptom.

The term “effective amount” used herein means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

The term “Administering” used herein refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion.

The terms “subject”, “individual”, and “patient” used herein are well known in the art and are used interchangeably herein to refer to any subjects, particularly mammals, in need of treatment subjects. Examples include, but are not limited to, humans and other primates, including non-human primates, such as chimpanzees and other ape and monkey species. The terms individual, subject and patient by themselves do not denote a particular age, sex, race, etc.

In the embodiments of method of this invention, the disease or condition is a disease or condition caused by infection of SARS-Cov-2 variants, such as a Delta variant, a Omicron variant or a Lambda variant.

In the tenth aspect, this invention provides the use of the recombinant S protein, mRNA, the composition, the recombinant plasmid, or mRNA-carrier particle of this invention in the preparation of medicament for preventing and/or treating a disease or condition associated with SARS-COV-2 infection in a subject.

In the eleventh aspect, this invention provides the recombinant S protein, mRNA, the composition, the recombinant plasmid, or mRNA-carrier particle of this invention for use in preventing and/or treating a disease or condition associated with SARS-COV-2 infection in a subject.

In the use embodiments of this invention, the disease or condition is a disease or condition caused by infection of SARS-COV-2 variants such as a Delta variant, a Omicron variant or a Lambda variant.

### BRIEF DESCRIPTION OF DRAWINGS

FIG. 1 shows a schematic diagram of RBMRNA-Delta plasmid.

FIG. 2 shows electrophoresis result diagram of mRNA with nucleic acid sequence of SEQ ID NO. 14, which was obtained by transcribing from RBMRNA-Delta plasmid.

FIG. 3 shows Western blot result diagram of recombinant S protein expressed from RBMRNA-Delta mRNA.

FIG. 4 shows result of inducing T lymphocytes to secrete IFN-γ, IL-2, IL-4 and IL-5 by RBMRNA-Delta 1 vaccine in mice, as measured by ELISPOT, *: P<0.05, **: P<0.01.

FIG. 5 shows result of inducing T lymphocytes to secrete IFN-γ, II-2, II-4 and II-5 by RBMRNA-Delta 2 vaccine in mice, as measured by ELISPOT, *: P<0.05, **: P<0.01.

FIG. 6 shows result of inducing T lymphocytes to secrete IFN-γ, IL-2, IL-4 and IL-5 by RBMRNA-Delta 3 vaccine in mice, as measured by ELISPOT, *: P<0.05, **: P<0.01.

FIG. 7 shows secretion result of cytokines, IFN-γ, IL-2, IL-4 and II-5 induced by RBMRNA-Delta 1 vaccine in mice, as measured by flow cytometry, *: P<0.05, **: P<0.01.

FIG. 8 shows secretion result of cytokines, IFN-γ, IL-2, IL-4 and IL-5 induced by RBMRNA-Delta 2 vaccine in mice, as measured by flow cytometry, *: P<0.05, **: P<0.01.

FIG. 9 shows secretion result of cytokines, IFN-γ, II-2, IL-4 and II-5 induced by RBMRNA-Delta 3 vaccine in mice, as measured by flow cytometry, *: P<0.05, **: P<0.01.

FIG. 10 shows immune response result of T cell subsets induced by RBMRNA-Delta 1 vaccine in mice, as measure by flow cytometry, *: P<0.05, **: P<0.01, ***: P<0.001.

FIG. 11 shows immune response result of T cell subsets induced by RBMRNA-Delta 2 vaccine in mice, as measure by flow cytometry, *: P<0.05, **: P<0.01.

FIG. 12 shows immune response result of T cell subsets induced by RBMRNA-Delta 3 vaccine in mice, as measure by flow cytometry, *: P<0.05, **: P<0.01, ***: P<0.001.

FIG. 13 shows result of serum IgG antibody level in mice after vaccination with RBMRNA-Delta 1 vaccine.

FIG. 14 shows result of serum IgG antibody level in mice after vaccination with RBMRNA-Delta 2 vaccine.

FIG. 15 shows result of serum IgG antibody level in mice after vaccination with RBMRNA-Delta 3 vaccine.

FIG. 16 shows serum neutralizing effect against wild type pseudovirus and Delta type pseudovirus after vaccination with RBMRNA-Delta 1 vaccine.

FIG. 17 shows serum neutralizing effect against wild type pseudovirus and Delta type pseudovirus after vaccination with RBMRNA-Delta 3 vaccine.

FIG. 18 shows a schematic diagram of RBMRNA-Omicron plasmid.

FIG. 19 shows mRNA electrophoresis result diagram of RBMRNA-Omicron mRNA transcribed from RBMRNA-Omicron plasmid.

FIG. 20 shows Western blot result diagram of recombinant S protein expressed from RBMRNA-Omicron mRNA.

FIG. 21 shows result of inducing T lymphocytes to secrete cytokine IFN-γ, IL-2, IL-4 and IL-5 by RBMRNA-Omicron vaccine in vivo in mice, as measured by ELISPOT, *: P<0.05, **: P<0.01, ***: P<0.001, ****: P<0.0001.

FIG. 22 shows secretion result of cytokine IFN-γ, IL-2, IL-4 and IL-5 induced by RBMRNA-Omicron vaccine in vivo in mice, as measured by flow cytometry, *: P<0.05, **: P<0.01.

FIG. 23 shows immune response result of T cell subsets induced by RBMRNA-Omicron vaccine in vivo in mice, as measure by flow cytometry, *: P<0.05, **: P<0.01, ***: P<0.001, *** P<0.0001.

FIG. 24 shows result of serum IgG antibody level in mice after vaccination with RBMRNA-Omicron vaccine or RBMRNA-combined vaccine.

FIG. 25 shows result of neutralization antibody (NAb) titer against Omicron type live virus in mice after vaccination with RBMRNA-combined vaccine, *: P<0.05, **: P<0.01, ***: P<0,001, ****: P<0.0001.

FIG. 26 shows result of TCID50 of Delta type virus in mice after vaccination with RBMRNA-Omicron vaccine or RBMRNA-combined vaccine, *: P<0.05, **: P<0.01, ***: P<0.001, ****: P<0.0001.

FIG. 27 shows result of TCID50 of Omicron type virus in mice after vaccination with RBMRNA-Omicron vaccine or RBMRNA-combined vaccine, *: P<0.05, **: P<0.01, ***: P<0.001, ****: P<0.0001, #: no significance.

### EXAMPLES

This invention is further described in the following examples, the advantages and features of this invention will be clearer with the description. It should be understand that these examples are only used for explaining this invention but not for limiting the scope of this invention described herein. The following examples do not specify the specific conditions of experimental methods, according to conventional conditions in this field, such as conditions described in Sambrook and Russeii et al., Molecular Cloning: A Laboratory Manual (Third Edition) (2001) CSHL Press, or conditions suggested by manufacturer. Unless otherwise defined, or the used experimental materials and reagents in following examples could be purchased commercially.

### Example 1. Preparation of mRNA

Preparation of RBMRNA-Delta mRNA

Based on wild type SARS Cov-2 S protein, the recombinant S protein (SEQ ID NO. 3) was obtained after subjecting to the following mutations: T19R, G142D, E156G, F157 deletion, R158 deletion, A222V, 1,452R, T478K, D614G, P681R, D950N; RRAR at positions 682-685 (S1/S2 cleavage site) were mutated to GGSG; KR at positions 814-815 (S2 cleavage site) were mutated to AN; KV sequence at positions 986-987 were mutated to two prolines PP.

DNA coding sequence (SEQ ID NO. 11) was designed based on the recombinant S protein sequence. After adding such as 5′-UTR, 3′-UTR, polyA sequence to DNA coding sequence, inserting it into pT7TS plasmid by homologous recombination for construction, forming a recombinant vector pT7TS-2.0 and obtaining final recombinant plasmid which was named as RBMRNA-Delta plasmid.

Elements contained in the RBMRNA-Delta plasmid comprised original sequence (SEQ ID NO. 6), 17 promoter sequence (SEQ ID NO. 7), 5′-UTR sequence (SEQ ID NO. 8), 3′-UTR sequence (SEQ ID NO. 10), 3′ end poly adenylate (polyA) sequence, ampicillin resistance gene promoter, kanamycin sulfate resistance gene. The stability, translation efficiency and immunogenicity of mRNA transcribed by RBMRNA-Delta plasmid were regulated by these non-coding structures.

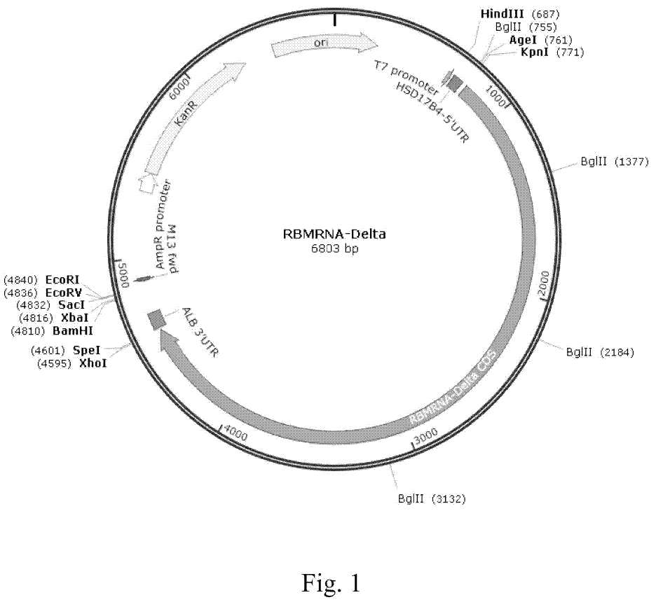

To sequence the coding region and polyA region of RBMRNA-Delta plasmid, the inserted target gene sequence was completely the same with the reference sequence, the entire successfully constructed plasmid structure was shown in FIG. 1, RBMRNA-Delta plasmid was transcribed in vitro to obtain mRNA (named RBMRNA-Delta mRNA), mRNA was translated to obtain protein (named RBMRNA-Delta protein).

The mRNA sequence transcribed by recombinant plasmid was shown in SEQ ID NO. 14. The size and integrity of the mRNA obtained by transcription were analyzed by Agilent 2200 Tapestation automatic electrophoresis system. The result was shown in FIG. 2, the transcribed mRNA showed a single band and no degradation.

mRNAs as shown in SEQ ID NO. 15 and SEQ ID NO. 16 were obtained by the above mentioned method, the DNA coding sequences thereof were shown in SEQ ID NO. 12 and SEQ ID NO. 13 respectively, the amino acid sequences of encoded recombinant S protein were shown in SEQ ID NO. 5 and SEQ ID NO. 4 respectively. Compared with the wild type S protein, the obtained recombinant S protein comprised the following mutations:
(1) T19R, G142D, E156G, F157 deletion, R158 deletion, A222V, L452R, T478K, D614G, P681R, D950N; RRAR at position 682-685 (S1/S2 cleavage site) were mutated to GGSG; KR at position 814-815 (S2 cleavage site) were mutated to AN; transmembrane domain deletion and cytoplasmic domain deletion; fusion peptide domain deletion; T4 phage fibritin motif was connected to C terminal of extracellular domain; the signal peptide sequence was replaced with immunoglobulin heavy chain variable region (IGHIV) signal sequence (SEQ ID NO. 4);
(2) KV at position 986-987 were mutated to two prolines PP (SEQ ID NO. 5) on the basis of the above (1).
   Preparation of RBMRNA-Omicron mRNA

Based on wild type SARS Cov-2 S protein, the recombinant S protein (SEQ ID NO. 25) was obtained after subjecting to the following mutations: A67V, H69del, V70del, T95I, G142del, V143del, Y144 del, Y145D, N211del, L212I and insertion mutations of three amino acids E, P, E between R214 and D215, G339D, S371L, S373P, S375F, K417N, N440K, G446S, S477N, T478K, F484A, Q493R, G496S, Q498R, N501Y, Y505H, T547K, D614G, H655Y, N679K, P681H, N764K, D796Y, N856K, Q954H, N969K, 1981F mutation; RRAR at positions 682-685 (S1/S2 cleavage site) were mutated to GGSG; KR at positions 814-815 (S2 cleavage site were mutated to AN); KV at positions 986-987 were mutated to two proline PP.

DNA coding sequence (SEQ ID NO. 26) was designed based on the recombinant S protein sequence. After adding such as 5′-UTR, 3′-UTR, polyA sequence to DNA coding sequence, inserting it into pT7TS plasmid by homologous recombination for construction, forming a recombinant vector pT7TS-2.0 and obtaining final recombinant plasmid (named RBMRNA-Omicron plasmid, with nucleic acid sequence of SEQ ID NO. 28).

Elements contained in the RBMRNA-Omicron plasmid comprised original sequence (SEQ ID NO. 6), 17 promoter sequence (SEQ ID NO. 7), 5′-UTR sequence (SEQ ID NO. 8), 3′-UTR sequence (SEQ ID NO. 10), 3′ end poly adenylate (poly A) sequence, ampicillin resistance gene promoter, kanamycin sulfate resistance gene. The stability, translation efficiency and immunogenicity of the mRNA transcribed by RBMRNA-Omicron plasmid was regulated by these non-coding structures.

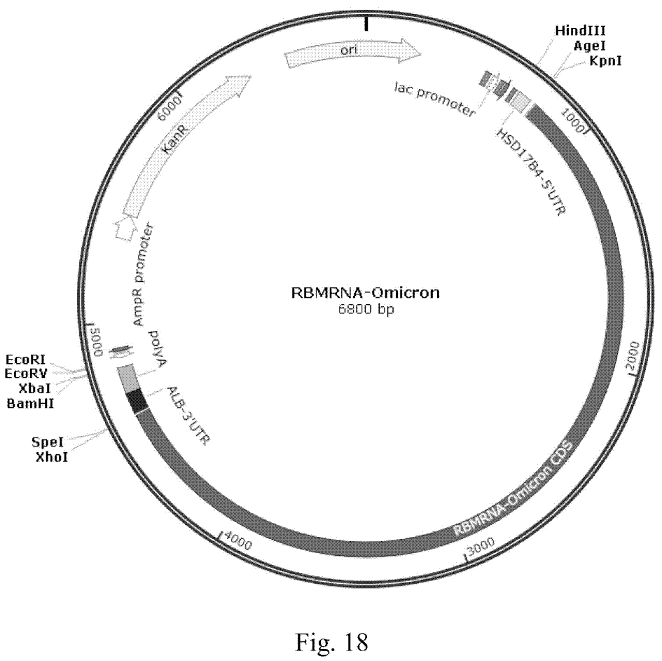

To sequence the coding region and poly A region of RBMRNA-Omicron plasmid, the inserted target gene sequence was completely the same with the reference sequence, the successfully constructed entire plasmid structure was shown in FIG. 18, RBMRNA-Omicron plasmid (SEQ ID NO. 28) was transcribed in vitro to obtain a mRNA (named RBMRNA-Omicron mRNA) (SEQ ID NO. 27), the mRNA was translated to obtain a protein (named RBMRNA-Delta protein) (SEQ ID NO. 25).

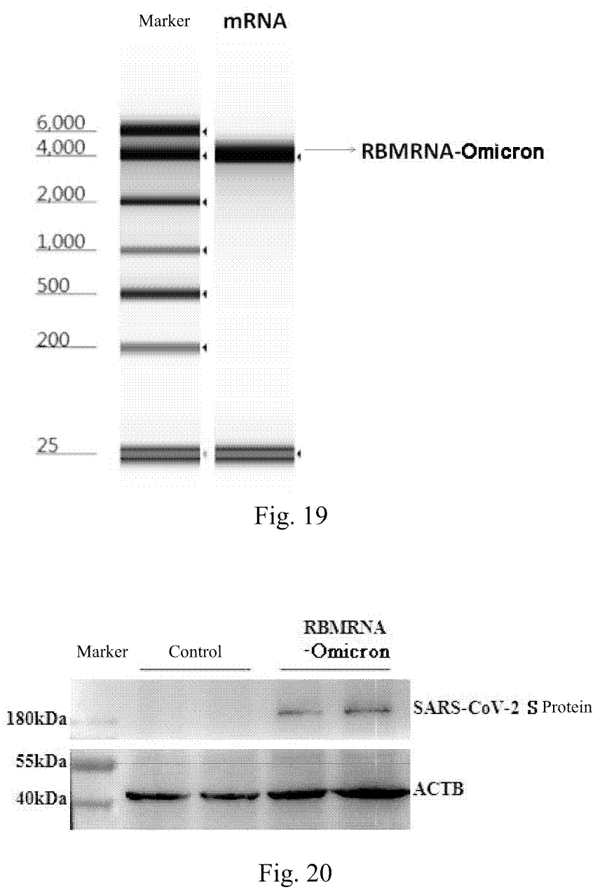

The size and integrity of the mRNA obtained by transcription of recombinant plasmid RBMRNA-Omicron plasmid was analyzed by Agilent 2200 Tapestation automatic electrophoresis system, the result showed that the transcribed mRNA had a single band and no degradation (FIG. 19).

### Example 2. Expression and Verification of mRNA

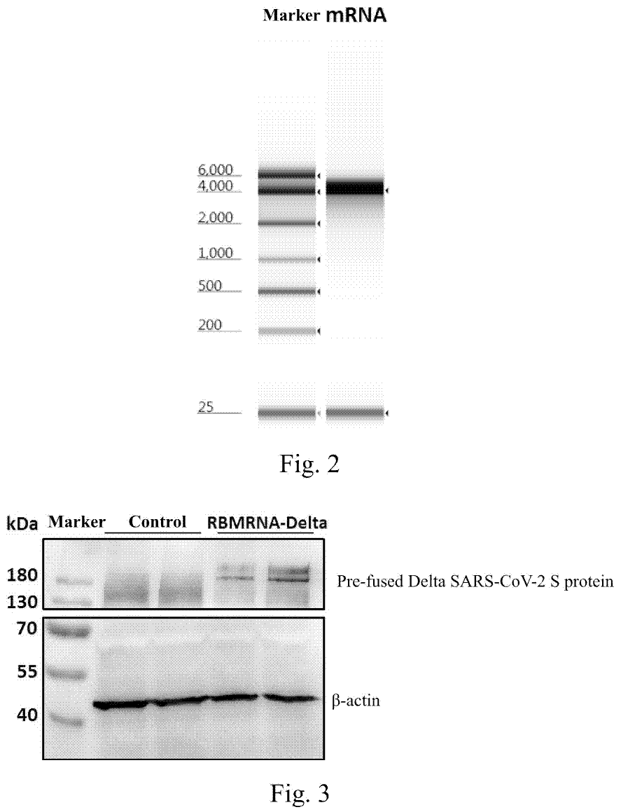

Referring to the manual of Lipofectamine MessagerMAX (ThermoFisher Scientific), 2.5 μg RBMRNA-Delta mRNA (SEQ ID NO. 14) and RBMRNA-Omicron mRNA (SEQ ID NO. 27) obtained from example 1 were used to transfect 293T cells respectively, untransfected cells were used as negative control, 24 hours after transfection, the expression of Delta type or Omicron type SARS-COV-2 pre-fusion S protein were assayed by Western blot, wherein an rabbit-anti-SARS-COV-2 S protein antibody (GeneTex, GTX632604) was used for Western blot and a goat-anti-rabbit-HRP secondary antibody were used for labeling. The results were shown in FIG. 3 (Delta type) and FIG. 20 (Omicron type), the expression of pre-fusion S protein with the same size as expected was successfully detected at 180 kDa.

### Example 3, Th1 or Th2 Immune Response Induced by mRNA Vaccines Preparation of mRNA Vaccines

Three RBMRNA-Delta mRNA sequences (SEQ ID NO. 14-16) and RBMRNA-Omicron mRNA sequence (SEQ ID NO. 27) of example 1 were used to prepare mRNA vaccines respectively, named RBMRNA-Delta 1 vaccine, RBMRNA-Delta 2 vaccine, RBMRNA-Delta 3 vaccine and RBMRNA-Omicron vaccine respectively. Lipid nanoparticles comprising the following components were used to encapsulate mRNA: 8-(3-hydroxypropyl) (9,12-dienyl-octadecyl-1)-amino-octanoic acid heptadecane-9-ol ester, distearoylphosphatidylcholine (DSPC), 1,2-Dimyristoyl-rac-glycerol-3-methoxypolyethylene glycol 2000 (DMG-PEG2000) and cholesterol. Preparation method included dissolving the above mentioned components in ethanol solution, mixing the lipid ethanol solution and mRNA aqueous solution by micro fluidic mixer to obtain lipid nanoparticulars, and conducting dialysis, ultrafiltration and micron membrane filtration on the mixture to obtain mRNA-LNP vaccine preparations. The specific vaccine preparation method referred to such as Chinese patent application no, 202011369776.2, which is herein incorporated by reference in its entirety.

### Th1 or Th2 Immune Response Induced by RBMRNA-Delta Vaccine

Three RBMRNA-Delta mRNA vaccines obtained were used in BALB/c mice immune experiments. Immunized mice were 6-8 weeks old female SPF grade healthy BALB/c mice. Mice were evenly and randomly divided into solvent control group (PBS), low dose vaccine group (1 μg/mouse), medium dose vaccine group (5 μg/mouse) and high dose vaccine group (20 μg/mouse) according to the mice's weight, 12 mice each group. After grouping, mice were inoculated with the vaccine preparations twice on Day 0 and Day 14 by intramuscular injection of the set doses to get prime immunization and boost immunization, respectively, the solvent control group was administered with an equal volume of PBS.

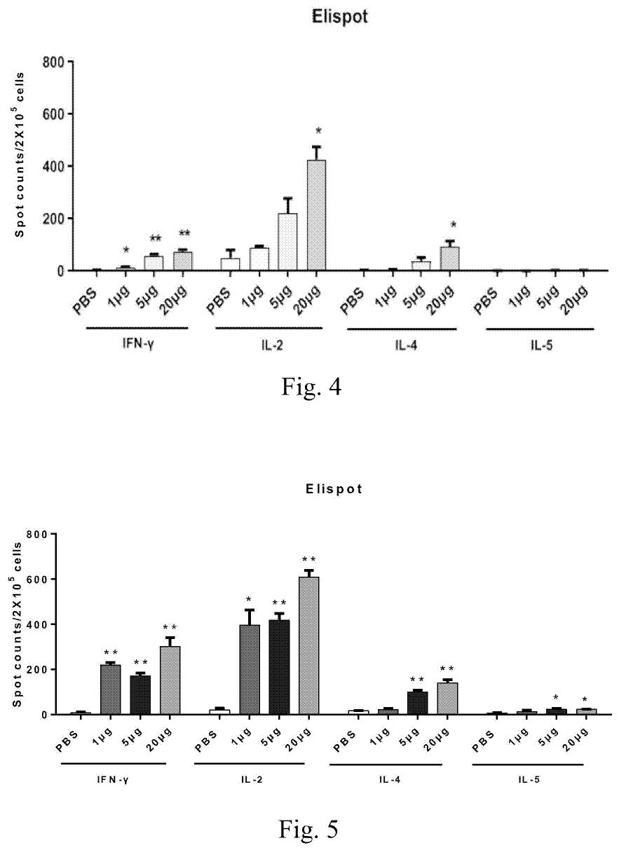

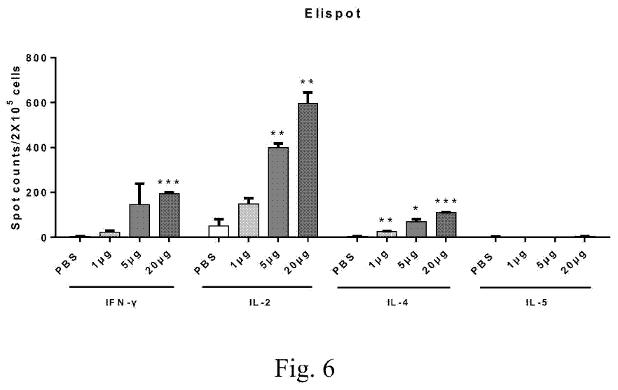

7 days after boost immunization, the mouse spleens were collected to separate splenic lymphocytes, T lymphocytes which secreted INF-γ, IL-2, IL-4 and IL-5 were detected by the ELISPOT method. The results of RBMRNA-Delta 1 vaccine, RBMRNA-Delta 2 vaccine and RBMRNA-Delta 3 vaccine were shown in FIG. 4-6 respectively. These results showed that T lymphocytes secreting Th1-type cytokines INF-γ and II-2 were obviously more than T lymphocytes secreting Th2-type cytokines IL-4 and IL-5 after vaccination with 3 vaccines of low, medium and high doses.

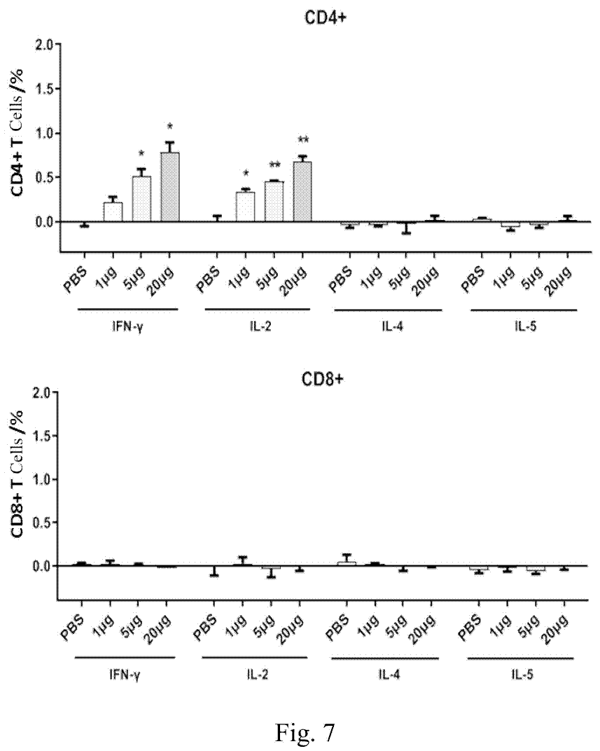

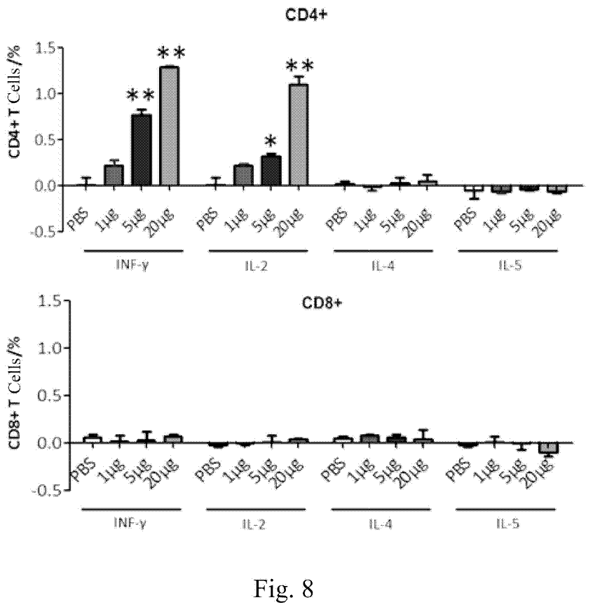

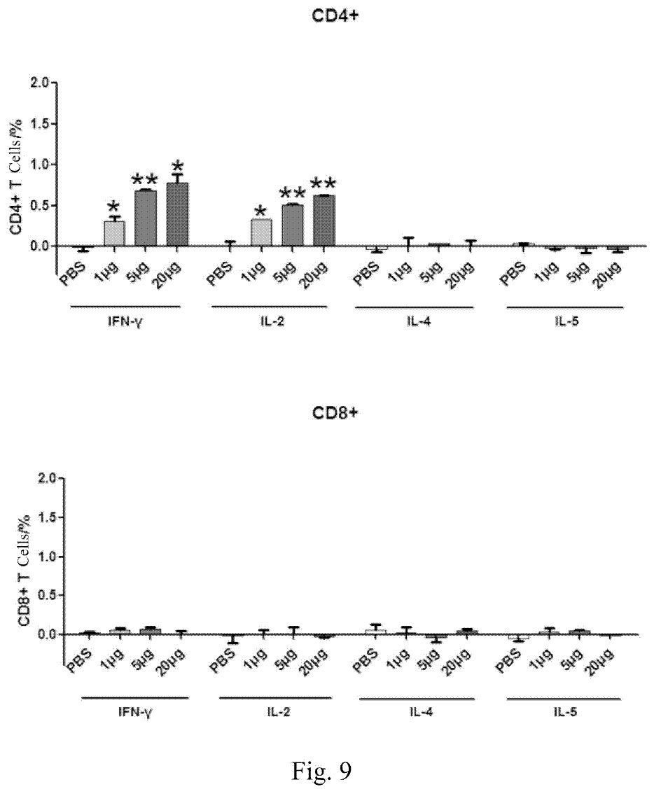

9 days after boost immunization, anti-IFN-γ antibody (Biolegend, 505808), anti-II-2 antibody (Biolegend, 503808), anti-II-4 antibody (Biolegend, 504104) and anti-IL-5 body (Biolegend, 504304) were used to detect the levels of cytokines INF-γ, IL-2, IL-4 and IL-5 level by flow cytometry, for further evaluating Th1 or Th2 immune response induced by mRNA vaccines. The results of RBMRNA-Delta 1 vaccine, RBMRNA-Delta 2 vaccine and RBMRNA-Delta 3 vaccine were shown in FIG. 7-9 respectively. These results showed that all these 3 vaccines caused a dose dependent increase of the levels of Th1 type cytokines INF-γ and IL-2 in CD4+ T cell, while the level of cytokines in CD8-T cell did not change significantly.

These results showed that immune response induced by 3 RBMRNA-Delta vaccines were Th1 type bias immune response.

### Th1 or Th2 Immune Response Induced by RBMRNA-Omicron Vaccine

RBMRNA-Omicron vaccine was used in BALB/c mice immune experiment. Immunized mice were SPF grade healthy BALB/c mice (6-8 weeks old, female). The mice were evenly and randomly divided into solvent control group (PBS), RBMRNA-Omicron vaccine low dose group (1 μg/mouse) and RBMRNA-Omicron vaccine high dose group (20 μg/mouse) according to the mice's weight, 3 mice each group. After grouping, the mice were inoculated with the vaccine twice by intramuscular injection of the set doses on Day 0 and Day 21 to get prime immunization and boost immunization, respectively, the solvent control group was administered with an equal volume of PBS.

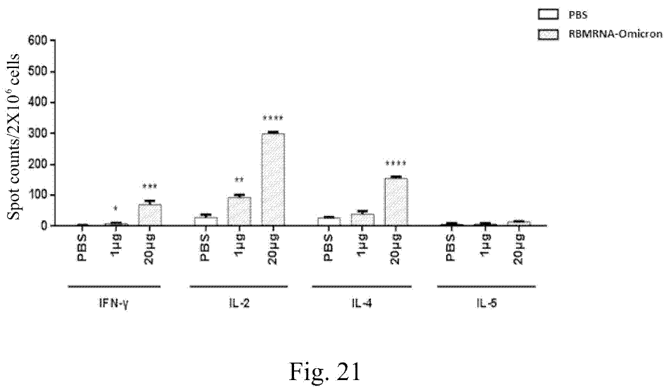

7 days after boost immunization, SARS-COV-2 S protein was used as irritant to stimulate the separated mouse splenic lymphocytes, detected the counting of T lymphocytes that secrete cytokines INF-γ, IL-2, IL-4 and IL-5 by ELISPOT method. As shown in FIG. 21, compared with solvent control group (PBS), both low and high dose RBMRNA-Omicron vaccines obviously increased the counting of T lymphocytes that secrete Th1 type cytokines INF-γ, IL-2 and Th2 type cytokine IL-4, but there was not an obvious change of the counting of T lymphocytes that secrete Th2 type cytokine IL-5.

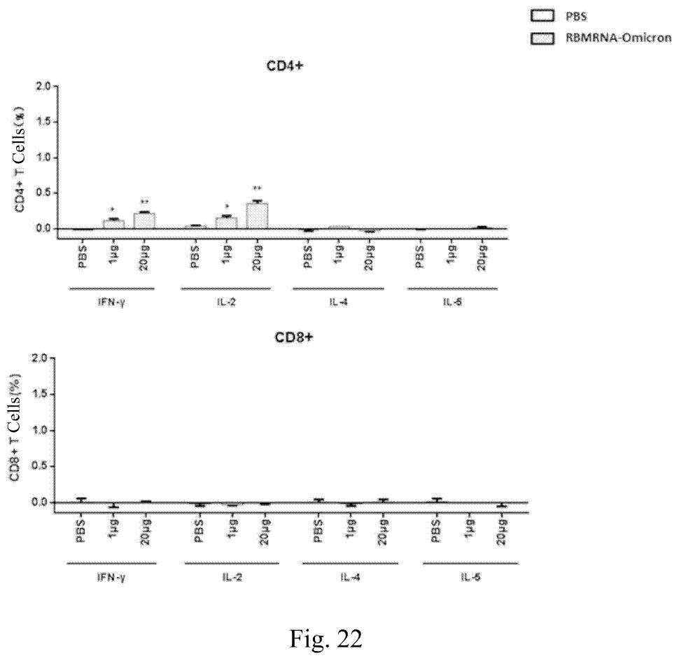

9 days after the boost immunization, anti-IFN-γ antibody (Biolegend, 505808), anti-II-2 antibody (Biolegend, 503808), anti-II-4 antibody (Biolegend, 504104) and anti-IL-5 body (Biolegend, 504304) were used to detect the levels of cytokines INF-γ, IL-2, IL-4 and IL-5 by flow cytometry respectively, for further evaluating Th1 or Th2 immune response induced by RBMRNA-Omicron vaccine. As shown in FIG. 22, RBMRNA-Omicron vaccine caused a dose dependent increase of the level of Th1 type cytokines INF-γ and II-2 in CD4+ T cell, while the level of cytokines in CD8+ T cell did not change significantly.

These results showed that immune response induced by RBMRNA-Omicron vaccine was Th1 type bias immune response.

### Example 4. Detection of T Cell Subsets

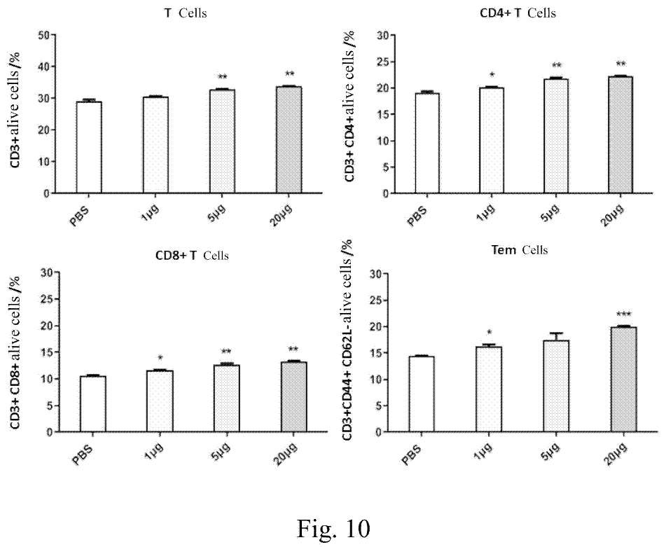

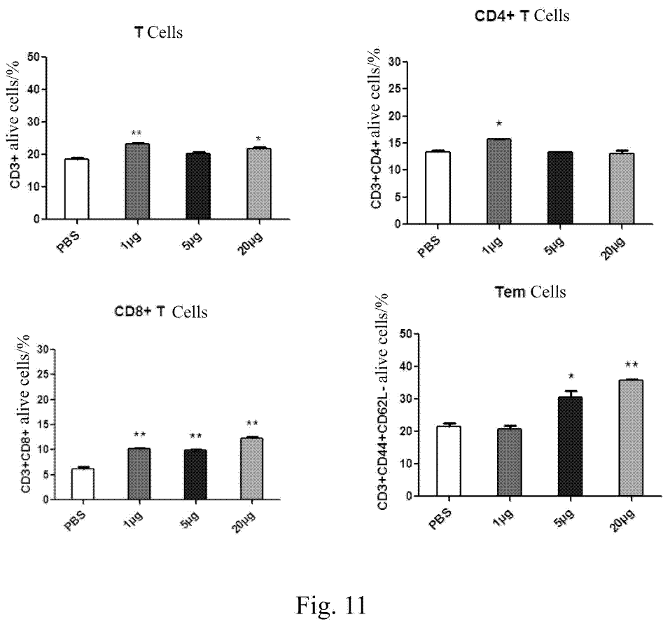

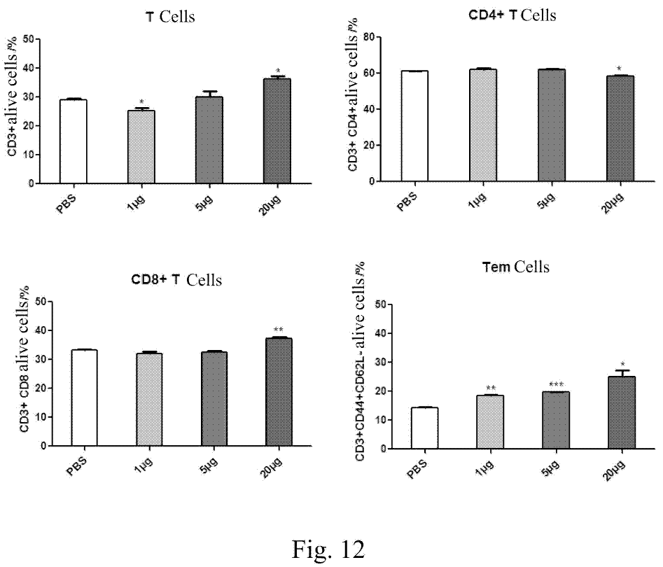

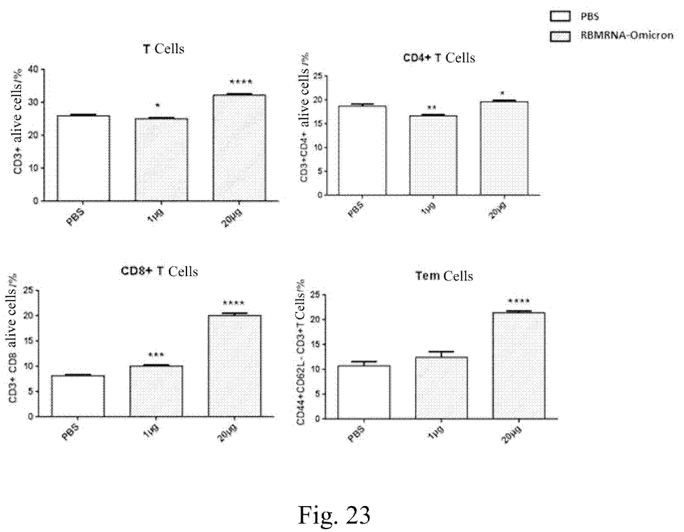

Using the same experimental method as in Example 3, the mice were immunized by 3 RBMRNA-Delta vaccines and RBMRNA-Omicron vaccine, 3 mice each group. The separated mouse splenic lymphocytes were used in immune experiment, 9 days after boost immunization, T cell subsets were detected by flow cytometry for evaluating immune response level of T lymphocytes, CD4-T cells, CD8+ T cells, effector memory T (Tem) cells induced by RBMRNA-Delta vaccines and RBMRNA-Omicron vaccine. The results of RBMRNA-Delta 1 vaccine, RBMRNA-Delta 2 vaccine and RBMRNA-Delta 3 vaccine were shown in FIG. 10-12; the result of RBMRNA-Omicron vaccine was shown in FIG. 23.

These results showed that compared with solvent control group (PBS), all the 3 RBMRNA-Delta vaccines and RBMRNA-Omicron vaccine could induce the body to produce CD4+ T cells and CD8+ T cells mediated cell immune response, producing specific effector memory T cells and making the body to get immune memory protection.

### Example 5. Evaluation of IgG Antibody Titer Induced by mRNA Vaccine

### RBMRNA-Delta Vaccine Induced IgG Antibody Titer

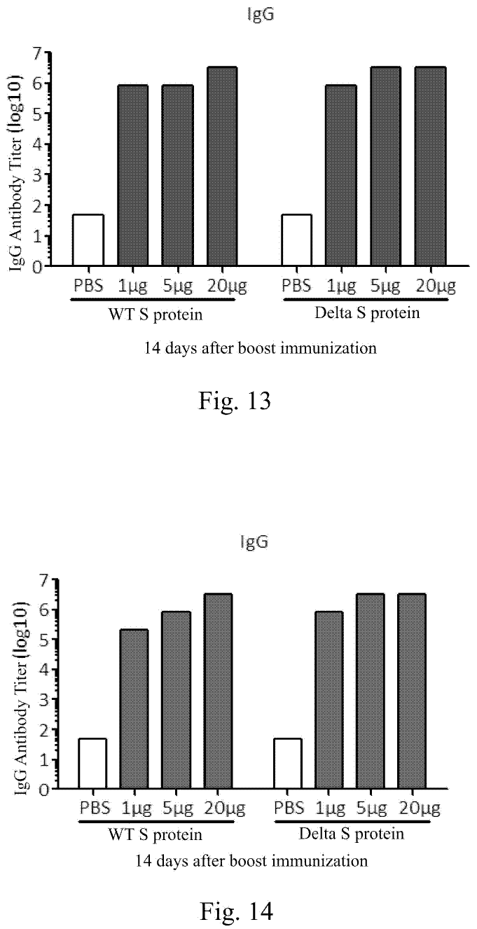

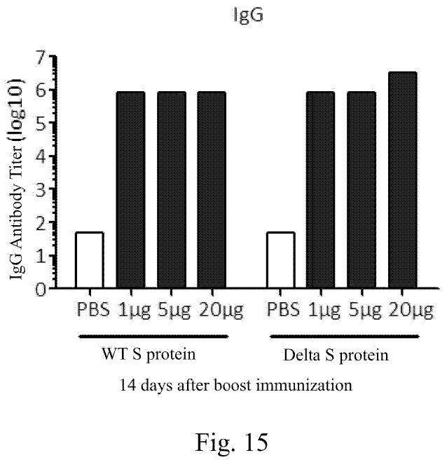

A similar experimental method as in Example 3 was applied, 3 RBMRNA-Delta mRNA vaccines were used in BALB/c mice immune experiment, and serum of 6 mice were collected in each group, 14 days after boost immunization, specific IgG antibody level in mouse serum were detected by indirect ELISA assay. The results of RBMRNA-Delta 1 vaccine, RBMRNA-Delta 2 vaccine and RBMRNA-Delta 3 vaccine were shown in FIG. 13-15 respectively.

IgG antibody level detection results showed that all the 3 vaccines at low, medium and high doses could induce high titer of IgG antibody in vivo in mice.

### RBMRNA-Omicron Vaccine and RBMRNA-Combined Vaccine Induced IgG Antibody Titer

Referring to the method in Example 3 to prepare RBMRNA-combined vaccine, the RBMRNA-combined vaccine comprised RBMRNA-Omicron mRNA (SEQ ID NO. 27) and RBMRNA-Delta mRNA (SEQ ID NO. 14), the molar ratio of these 2 mRNAs was 1:1.

The obtained RBMRNA-combined vaccine and RBMRNA-Omicron vaccine were used in BALB/c mice immune experiment. Mice were evenly and randomly divided into solvent control group (PBS), RBMRNA-Omicron vaccine low dose group (1 μg/mouse), RBMRNA-Omicron vaccine high dose group (20 μg/mouse), RBMRNA-combined vaccine low dose group (1 μg/mouse) and RBMRNA-combined vaccine high dose group (20 μg/mouse) according to the mice's weight, 6 mice each group. After grouping, mice were inoculated with the vaccines twice on Day 0 and Day 21 by intramuscular injection of the set doses to get prime immunization and boost immunization, respectively, the solvent control group was administered with an equal volume of PBS. The mice's serum were collected for the experiment, 14 days after the boost immunization, specific IgG antibody titers in the mice's serum were detected by indirect ELISA method.

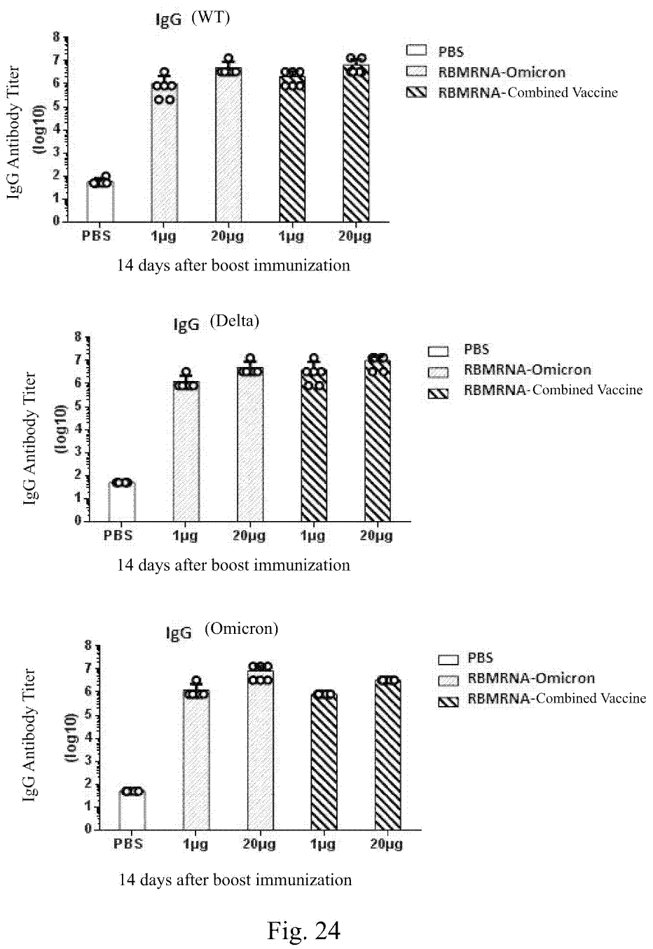

As shown in FIG. 24, both RBMRNA-Omicron vaccine and RBMRNA-combined vaccine could induce high titer of IgG antibodies in vivo in mice against wild type, delta type and omicron type SARS-COV-2.

### Example 6. Evaluation of Neutralizing Antibody Induced by RBMRNA-Delta mRNA Vaccines

A similar experiment as in Example 3 was applied, 3 RBMRNA-Delta mRNA vaccines were used in BALB/e mice immune experiment, serum of 6 mice were collected in each group. Vaccine group was only administered with high dose (20 μg/mouse). The mice's serum were collected on 14 days after prime immunization and 14 days after boost immunization respectively, S protein specific neutralizing antibodies were detected by pseudo virus neutralizing experiment. Specific procedures were performed below:

The serum was centrifuged after inactivation in water bath, then the supernatant was collected. The inactivated serum was diluted with serum-free DMEM medium. The diluted serum and pseudo virus were added into 96-well plate, and incubated together at 37° C., for 1 hour. After incubation, 293T-ACE2-p2A-mTagBFP2 cells were added into 96-well plate (these cells were obtained by in site knocking ACE2-p2A-mTagBFP2 into 293T cells according to CRISPR technology). Incubation was carried at 37° C., 5% CO₂ for 48 hours. After incubation, the plate was washed, then PBS and firefly luciferase substrate were added into the plate, luciferase chemiluminescence values were detected by multifunctional microplate reader after shaking in the dark. Reed-muench method was used to calculate neutralization titer.

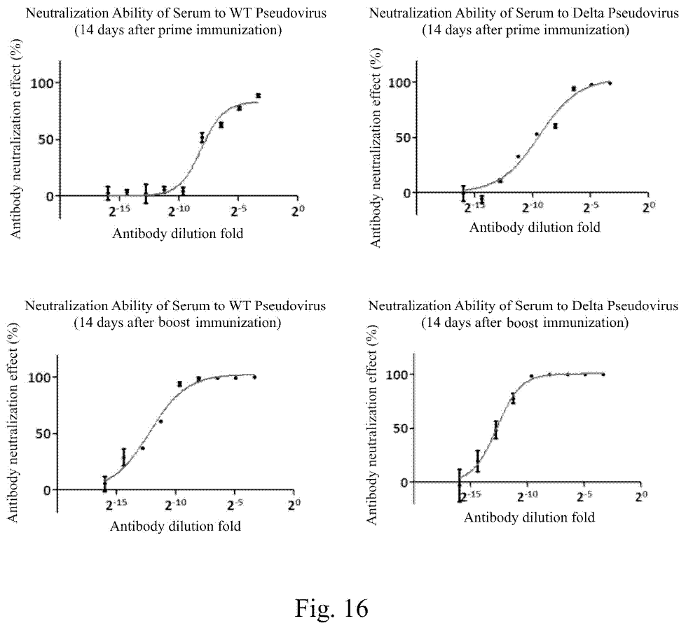

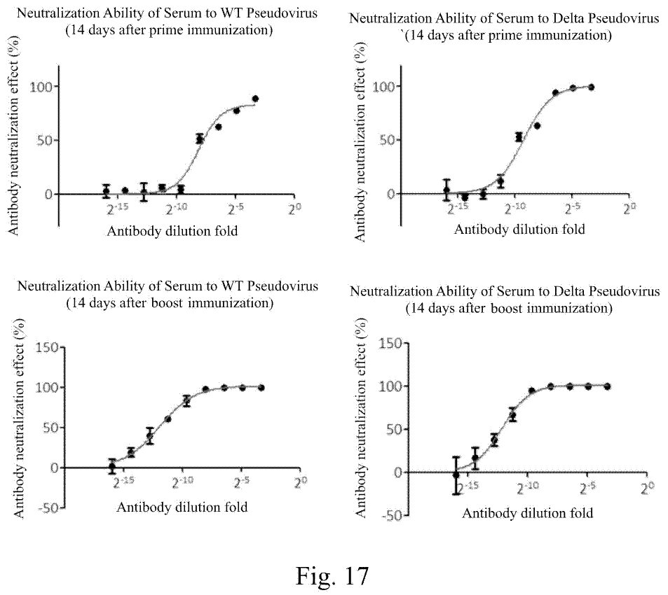

The antibody's neutralizing effect induced by RBMRNA-Delta 1 vaccine and RBMRNA-Delta 3 vaccine were shown in FIG. 16 and FIG. 17 respectively.

These results showed that all mouse serum with twice vaccination could basically completely neutralize delta type pseudo virus on 14 days after prime immunization, and basically completely neutralize wild type pseudo virus on 14 days after boost immunization. For RBMRNA-Delta 1 vaccine, the neutralizing titer of immunized mouse serum against wild type and delta type pseudo virus were 282 and 966 respectively, on 14 days after prime immunization; and were 4007 and 6903 respectively, on 14 days after boost immunization (FIG. 16). For RBMRNA-Delta 3 vaccine, the neutralizing titer of immunized mouse serum against wild type and delta type pseudo virus were 271 and 874 respectively, on 14 days after prime immunization; and were 4232 and 4624 respectively, on 14 days after boost immunization (FIG. 17).

### Example 7. Evaluation of Neutralizing Antibody Induced by RBMRNA-Delta 1, RBMRNA-Omicron and RBMRNA-Combined mRNA Vaccines

A similar experiment as in Example 3 was applied, RBMRNA-Delta 1 mRNA vaccine, RBMRNA-Omicron vaccine and RBMRNA-combined vaccine were used in BALB/e mice immune experiment, 6 mice each group, and the mouse serum were collected for the experiment. Vaccine group was only administered with high dose (20 μg/mouse). The mice's serum were collected on 14 days after boost immunization. Different vaccines were used in the neutralization experiment against different types of variant pseudo virus for evaluating the neutralization titer of different vaccines against different pseudo viruses. Specific procedures were performed below:

The serum was centrifuged after inactivation in water bath, then the supernatant was collected. The inactivated serum was diluted with serum-free DMEM medium. The diluted serum and pseudo virus were added into 96-well plate, and incubated together at 37° C., for 1 hour. After incubation, 293T-ACE2-p2A-mTagBFP2 cells were added into 96-well plate (these cells were obtained by in site knocking ACE2-p2A-mTagBFP2 into 293T cells according to CRISPR technology). Incubation was carried at 37° C., 5% CO₂ for 48 hours. After incubation, the plate was washed, then PBS and firefly luciferase substrate were added into the plate, luciferase chemiluminescence values were detected by multifunctional microplate reader after shaking in the dark. The specific procedures referred to such as Chinese patent application no, 202210019169.6, which is herein incorporated by reference in its entirety.

Neutralization titer was calculated by Reed-muench method.

The neutralization titers of antibodies induced by RBMRNA-Delta 1 vaccine, RBMRNA-Omicron vaccine and RBMRNA-combined vaccine against different pseudo viruses were shown in table 1,

TABLE 1
Neutralization titer of vaccines against different pseudo viruses

| | [symbol] | | | | | | | | | | | | | | | | | |
|---|---|---|---|---|---|---|---|---|---|---|---|---|---|---|---|---|---|---|
| [symbol] | [symbol] | | | | | | [symbol] | | | | | | [symbol] | | | | | |
| Wild type | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | | [symbol] |

[symbol]

indicates data missing or illegible when filed

As seen from table 1, RBMRNA-Delta 1 vaccine had strong inhibition effect against Delta type pseudo virus, while RBMRNA-Omicron vaccine had strong inhibition effect against Omicron type pseudo virus. Besides, RBMRNA-combined vaccine had strong inhibition effect against all of the wild type, Beta type, Gamma type, Alpha type, Delta type, Omicron type and Deltacron type pseudo viruses.

### Example 8. Live Virus Neutralization Assay

RBMRNA-combined vaccine was used in the BALB/c mice immune experiment as similar to that in Example 3, 6 mice each group, and the mice serum were collected for the experiment. Vaccine group were only administered with high dose (20 μg/mouse). The mice's serum were collected on 14 days after boost immunization. Serum samples collected from immunized mice were inactivated at 56° C., for 30 min and serially diluted with DMEM medium (GIBCO) in two-fold steps. The diluted serums were mixed with 100 TCID50 SARS-COV-2 live virus (Omicron, B.1.1.529) in 96-well plates at a ratio of 1:1 (vol/vol) and incubated at 37° C., for 1 hour. Then virus/serum mixtures were added to monolayers of Vero-E6 cells in 96-well plates in quadruplicate and the plates were incubated for 3-5 days at 37° C., in a 5% CO₂ incubator. Cytopathic effect (CPE) of each well was recorded under microscope, and the 50% neutralization Ab (NAb) titers were calculated.

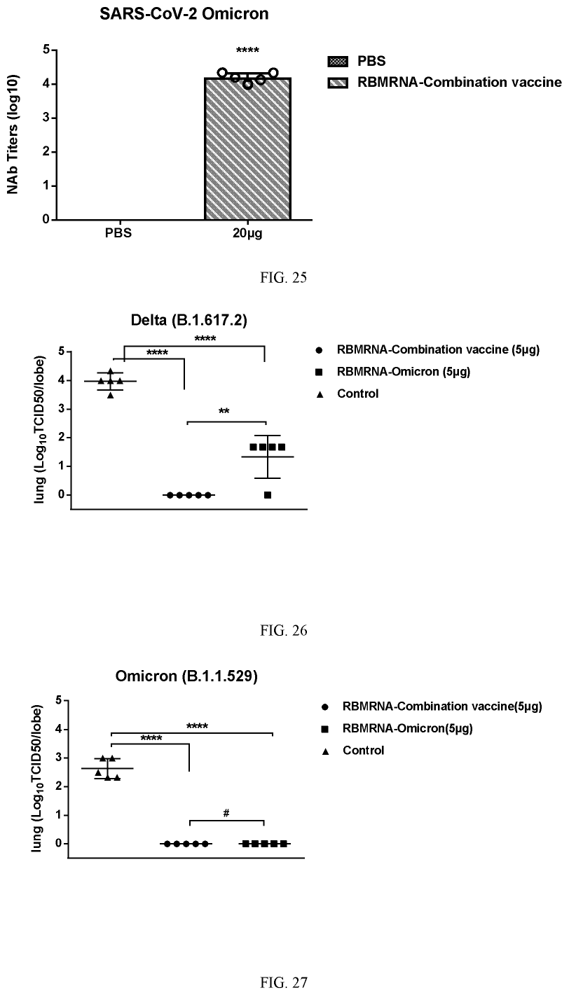

As shown in FIG. 25, compared with the PBS control group, mouse serum immunized with RBMRNA-combined vaccine has significantly improved neutralization effect against SARS-COV-2 Omicron type live virus, suggesting that RBMRNA-combined vaccine could inhibit Omicron type live virus effectively.

### Example 9. Virus TCID50 Assay

RBMRNA-Omicron vaccine and RBMRNA-combined vaccine were used in a TCID50 assay, 5 K18-hACE2 mice each group. Mice were intramuscularly vaccinated twice with 5 μg doses of RBMRNA-Omicron vaccine or RBMRNA-combined vaccine on Day 0 (as prime immunization) and Day 21 (as boost immunization). The control group was administered with an equal volume of PBS, 11 days after the boost immunization, mice were challenged with 1×10³ plaque-forming units (PFU) of Delta (B.1.617.2) live virus, 5 days after the infection, viral titers in right lungs of mice were quantified by TCID50 assay, and the results were shown in FIG. 26, 31 days after the boost immunization, mice were challenged with 1×10⁴ PFU of Omicron (B.1.1.529) live virus, 5 days after the infection, viral titers in right lungs of mice were quantified by TCID50 assay, and the results were shown in FIG. 27.

As seen from FIG. 26, compared with the control group, both the RBMRNA-Omicron vaccine and the RBMRNA-combined vaccine result in decreased viral titers for Delta live virus. As seen from FIG. 27, compared with the control group, both the RBMRNA-Omicron vaccine and the RBMRNA-combined vaccine result in decreased viral titers for Omicron live virus.

These results indicate that compared with the control group, both the RBMRNA-Omicron vaccine and the RBMRNA-combined vaccine could protect mice from infecting by Delta type and Omicron type SARS-COV-2 effectively.

This application refers to various issued patents, published patent applications, journal articles, and other publications, all of which are incorporated herein by reference. If any of the references cited conflict with this description, the present specification shall control. In addition, any particular embodiment of the present disclosure that falls within the purview of the prior art may be expressly excluded from any one or more of the claims. As the described embodiments are to be considered as known to those skilled in the art, they can be excluded, even if the exclusion is not explicitly listed in this application. Any particular embodiment of the present disclosure may be excluded from any claim for any reason in the presence or absence of the prior art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims,

What is claimed is:

1. A recombinant SARS-CoV-2 spike protein (S protein),
(a) comprising following mutations in an extracellular domain, compared with a wild type S protein;
T19R, G142D, E156G F157 deletion, R158 deletion, A222V, L452R, T478K, D614G, P681R and D950N;
wherein the amino acid positions are numbered according to the amino acid sequence of the wild type S protein as shown in SEQ ID NO. 29;
(b) comprising following mutations in an extracellular domain, compared with a wild type S protein;
A67V, H69 deletion, V70 deletion, T95I, G142 deletion, V143 deletion, Y144 deletion, Y145D, N211 deletion, L212I, insertion mutation of three amino acids E, P, E between R214 and D215, G339D, S371L, S373P, S375F, K417N, N440K, G446S, H655Y, N679K, P681H, N764K, D796 Y, N856K, Q954H, N969K and L981F;
wherein the amino acid positions are numbered according to the amino acid sequence of the wild type S protein as shown in SEQ ID NO. 29.

2. The recombinant S protein of claim 1, wherein a S1/S2 cleavage site RRAR and/or a S2 cleavage site KR are(is) mutated to lose the ability of being cleaved by Furin-like protease and lysosomal protease; preferably, the S1/S2 cleavage site is mutated to GGSG, and/or the S2 cleavage site is mutated to AN.

3. The recombinant S protein of claim 1, further comprising K986P and V987P mutations.

4. The recombinant S protein of claim 1, wherein in (a), the recombinant S protein does not comprise a fusion peptide (FP) domain; optionally, the recombinant S protein does not comprise a transmembrane domain and a cytoplasmic domain; optionally, the recombinant S protein further comprises a trimer domain facilitating the recombinant S protein to form a trimer, when being expressed; preferably, the trimer domain is a T4 phage fibritin trimer motif; optionally, the recombinant S protein further comprises a signal sequence, preferably, the signal sequence is a signal sequence of an immunoglobulin heavy chain variable region, or
wherein in (b), the recombinant S protein comprises, from N terminal to C terminal, an extracellular domain, a transmembrane domain and a cytoplasmic domain; preferably, the recombinant S protein further comprises a signal sequence, preferably, the signal sequence is a signal sequence of an immunoglobulin heavy chain variable region.

5. The recombinant S protein of claim 1, wherein,
in (a), the recombinant S protein has an amino acid sequence as shown in any any one selected from SEQ ID NO. 1-5, preferably has an amino acid sequence as shown in any one selected from SEQ ID NO. 3-5; or
in (b), the recombinant S protein has an amino acid sequence as shown in any one selected from SEQ ID NO. 20-25, preferably has an amino acid sequence as shown in SEQ ID NO. 25.

6-10. (canceled)

11. A mRNA encoding the recombinant S protein of claim 1.

12. The mRNA of claim 11, which comprises, from 5′ to 3′, a cap structure, 5′-UTR, open reading flame (ORF), 3′-UTR and a polyA tail;
preferably, wherein the 5′-UTR comprises a 5′-UTR derived from 17β-hydroxysteroid dehydrogenase 4 (HSD17B4) gene or homologs, fragments or variants thereof and/or a KOZAK se sequence, preferably the 5′-UTR comprises a sequence as shown in SEQ ID NO. 8 and/or SEQ ID NO. 9;
wherein the 3′-UTR comprises a 3′-UTR derived from albumin (ALB) gene or homologs, fragments or variants thereof, preferably, the 3′-UTR comprises a sequence as shown in SEQ ID NO. 10; and/or
wherein the polyA tail is 100-150 nucleotides in length.

13. (canceled)

14. The mRNA of claim 11, wherein the sequence of the mRNA is as shown in any one of SEQ ID NO. 14-16, or the sequence of the mRNA is as shown in SEQ ID NO. 27.

15. The mRNA of claim 11, wherein one or more nucleotides of the mRNA each is independently replaced by naturally occurring nucleotide analogues or artificially synthesized nucleotide analogues, wherein the naturally occurring nucleotide analogues are selected from pseudouridine, 2-thiouridine, 5-methyluridine, 5-methylcytidine and N6-methyladenosine and the artificially synthesized nucleotide analogues are selected from N1-methylpseudouridine and 5-ethynyluridine;
preferably, one or more uridine triphosphate of the mRNA each is independently replaced by pseudo-uridine triphosphate, 1-methyl-pseudo-uridine triphosphate or 5-ethynyl-uridine triphosphate, and/or one or more cytidine triphosphate each is independently replaced by 5-methyl-cytidine triphosphate.

16-20. (canceled)

21. A composition, which comprises the recombinant S protein in (a) of claim 1, and the recombinant S protein in (b) of a claim 1; or
wherein the composition comprises a mRNA encoding recombinant S protein in (a) of claim 1 and a mRNA encoding recombinant S protein in (b) of claim 1.

22. (canceled)

23. The composition of claim 21, wherein the composition comprises the mRNA having an amino sequence as shown in any one of SEQ ID NOs. 14-16 and the mRNA having an amino sequence as shown in SEQ ID NO. 27.

24. The composition of claim 21, wherein the molar ratio of the two kinds of recombinant S proteins or the two kinds of mRNAs in the composition is 1-3:1-3, preferably 1:1.

25. The composition of claim 21, which further comprises following recombinant S protein or mRNA encoding the same:
(a) a recombinant S protein comprising following mutations compared with a wild type S protein: K986P and V987P;
and/or
(b) a recombinant S protein comprising following mutations compared with a wild type S protein: G75V, T76I, R246 deletion, S247 deletion, Y248 deletion, L249 deletion, T250 deletion, P251 deletion, G252 deletion, D253N, L452Q, F490S, D614G, T859N; K986P; and V987P;
and/or
(c) a recombinant S protein comprising following mutations compared with a wild type S protein: mutation of a S1/S2 cleavage site to GGSG; K986P; and V987P;
and/or
(d) a recombinant S protein comprising following mutations compared with a wild type S protein: mutation of a S2 cleavage site to AN; K986P; and V987P;
and/or
(e) a recombinant S protein comprising following mutations compared with a wild type S protein: mutation of a S1/S2 cleavage site to GGSG; mutation of a S2 cleavage site to AN; K986P; and V987P;
and/or
(f) a recombinant S protein comprising following mutations compared with a wild type S protein: G75V, T76I, R246 deletion, S247 deletion, Y248 deletion, L249 deletion, T250 deletion, P251 deletion, G252 deletion, D253N, L452Q, F490S, D614G, T859N; mutation of a S1/S2 cleavage site to GGSG; K986P; and V987P;
and/or
(g) a recombinant S protein comprising following mutations compared with a wild type S protein: G75V, T76I, R246 deletion, S247 deletion, Y248 deletion, L249 deletion, T250 deletion, P251 deletion, G252 deletion, D253N, L452Q, F490S, D614G, T859N; mutation of a S2 cleavage site to AN; K986P; and V987P;
and/or
(h) a recombinant S protein comprising following mutations compared with a wild type S protein: G75V, T76I, R246 deletion, S247 deletion, Y248 deletion, L249 deletion, T250 deletion, P251 deletion, G252 deletion, D253N, L452Q, F490S, D614G, T859N; mutation of a S1/S2 cleavage site to GGSG; mutation of a S2 cleavage site to AN; K986P; and V987P.

26. A DNA encoding the mRNA of claim 11.

27. The DNA of claim 26, wherein the sequence of the DNA is as shown in any one of SEQ ID NOs. 11-13 and SEQ ID NO. 26.

28. A recombinant plasmid, which comprises the DNA of claim 26.

29. The recombinant plasmid of claim 28, wherein the recombinant plasmid is a pT7TS plasmid;
preferably, the recombinant plasmid further comprises an original sequence (Ori), a T7 promoter, 5′-UTR and 3′-UTR;
preferably, wherein the original sequence is of ColE1 type, preferably, the original sequence is as shown in SEQ ID NO. 6;
wherein the sequence of the T7 promoter is as shown in SEQ ID NO. 7;
wherein the 5′-UTR comprises a 5′-UTR derived from HSD17B4 or homologs, fragments or variants thereof, and/or a KOZAK sequence, preferably, the 5′-UTR comprises a sequence as shown in SEQ ID NO. 8 and/or SEQ ID NO. 9; and/or
wherein the 3′-UTR comprises a 3′-UTR derived from ALB or homologs, fragments or variants thereof, preferably, the 3′-UTR comprises a sequence as shown in SEQ ID NO. 10;
preferably, wherein the recombinant plasmid further comprises a polyA, a resistance gene promoter and a resistance gene;
preferably, the polyA is 100-150 nucleotides in length; the resistance gene promoter is an ampicillin resistance gene promoter; and/or the resistance gene is a kanamycin sulfate resistance gene;
more preferably, wherein the nucleic acid sequence of the recombinant plasmid is as shown in SEQ ID NO. 28.

30-33. (canceled)

34. A mRNA-carrier particle, which comprises a mRNA encoding the recombinant S protein in (a) of claim 1 and/or a mRNA encoding the recombinant S protein in (b) of claim 1, and a carrier material encapsulating the mRNA.

35. A method for preventing and/or treating a diseases or condition associated with SARS-CoV-2 infection in a subject, which comprises administering to the subject an effective amount of the recombinant S protein of claim 1,
a mRNA encoding the recombinant S protein in (a) or (b) of claim 1,
composition comprising the recombinant S protein in (a) of claim 1 and the recombinant S protein in (b) of claim 1,
a recombinant plasmid comprising a DNA encoding a mRNA that encodes the recombinant S protein in (a) of claim 1 or the recombinant S protein in (b) of claim 1, or
a mRNA-carrier particle comprising a mRNA encoding the recombinant S protein in (a) of claim 1 and/or a mRNA encoding the recombinant S protein in (b) of claim 1, and a carrier material encapsulating the mRNA.

36. The method of claim 35, wherein the disease or condition is a disease or condition caused by infection of SARS-CoV-2 variants, such as a Delta variant, a Omicron variant or a Lambda variant.


SEQUENCE LISTING (43 sequences)

SEQ ID NO: 1 - protein, 1271 aa, Artificial sequence
  Amino acid sequence of recombinant S protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1271 aa, Artificial sequence
  Amino acid sequence of recombinant S protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1271 aa, Artificial sequence
  Amino acid sequence of recombinant S protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1229 aa, Artificial sequence
  Amino acid sequence of recombinant S protein
MDWIWRILFLVGAATGAHSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 5 - protein, 1230 aa, Artificial sequence
  Amino acid sequence of recombinant S protein
MDWIWRILFLVGAATGAHSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 6 - DNA, 802 bp, Artificial sequence
  Original sequence
agtactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggtccctggccttttgctggccttttgctca

SEQ ID NO: 7 - DNA, 20 bp, Artificial sequence
  T7 promoter
taatacgactcactataatg

SEQ ID NO: 8 - DNA, 62 bp, Artificial sequence
  5′UTR
gtcccgcagtcggcgtccagcggctctgcttgttcgtgtgtgtgtcgttgcaggccttattc

SEQ ID NO: 9 - DNA, 24 bp, Artificial sequence
  kozak sequence
agatctaccggtggtaccgccacc

SEQ ID NO: 10 - DNA, 89 bp, Artificial sequence
  3′UTR
agccaacaccctgtctaaaaaacataaatttctttaatcattttgcctcttttctctgtgcttcaattaataaaaaatggaaagaacct

SEQ ID NO: 11 - DNA, 3816 bp, Artificial sequence
  DNA sequence of RBMRNA-Delta 1 vaccine
atgttcgtgttcctggtgctgctgcctctggtgagcagccagtgcgtgaatctgagaaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggacgtttattatcataagaacaacaagagctggatggagagcggagtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgtcctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaacctggactcgaaagttggaggcaattacaattaccgctacagactgttcagaaagagcaatctgaagcctttcgagagagacatcagcaccgagatctaccaggccggcagcaagccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggcaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccgcggcggcagcggcagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcgccaacagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcagaacgtggtgaatcagaatgcccaggccctgaataccctggtgaagcagctgagcagcaatttcggcgccatcagtagtgtactcaacgacatcctgagcagactggacccaccagaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctaa

SEQ ID NO: 12 - DNA, 3690 bp, Artificial sequence
  DNA sequence of RBMRNA-Delta 2 vaccine
atggactggatctggagaatcctgttcctggtgggggctgccactggcgcccatagccagtgcgtgaatctgagaaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggacgtttattatcataagaacaacaagagctggatggagagcggagtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgtcctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaacctggactcgaaagttggaggcaattacaattaccgctacagactgttcagaaagagcaatctgaagcctttcgagagagacatcagcaccgagatctaccaggccggcagcaagccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggcaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctcggggcagcggcagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagccagatccaagcaagcctagcgccaacagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcagaacgtggtgaatcagaatgcccaggccctgaataccctggtgaagcagctgagcagcaatttcggcgccatcagtagtgtactcaacgacatcctgagcagactggacccgccggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggcctagcgccatcggcggctacatccccgaggcccccagggacggccaggcctacgtgaggaaggacggcgagtgggtgctgctgagcaccttcctgtga

SEQ ID NO: 13 - DNA, 3816 bp, Artificial sequence
  DNA sequence of RBMRNA-Delta 3 vaccine
atgttcgtgttcctggtgctgctgcccctggtgagcagccagtgcgtgaacctgagaacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcggagtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagtcctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactaccgctaccggctgttccggaagagcaacctgaagcccttcgagcgggacatcagcaccgagatctaccaagccggctccaagccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacgcggcggcagcggcagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcgccaacagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcagaacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctga

SEQ ID NO: 14 - RNA, 4130 nt, Artificial sequence
  mRNA sequence of RBMRNA-Delta 1 vaccine
augagacaagcuugucccgcagucggcguccagcggcucugcuuguucgugugugugucguugcaggccuuauucagaucuaccggugguaccgccaccauguucguguuccuggugcugcugccucuggugagcagccagugcgugaaucugagaaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggacguuuauuaucauaagaacaacaagagcuggauggagagcggaguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcguccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccgcuacagacuguucagaaagagcaaucugaagccuuucgagagagacaucagcaccgagaucuaccaggccggcagcaagccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccagggcgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagccgcggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcagaacguggugaaucagaaugcccaggcccugaauacccuggugaagcagcugagcagcaauuucggcgccaucaguaguguacucaacgacauccugagcagacuggacccaccagaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugaccuccuguuguuccuguuugaaaggguguuguucguguggguccugcugcaaguucgacgaggacgacagcgagccugugcugaagggcgugaagcugcacuacaccuaacucgagacuaguagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 4004 nt, Artificial sequence
  mRNA sequence of RBMRNA-Delta 2 vaccine
augagacaagcuugucccgcagucggcguccagcggcucugcuuguucgugugugugucguugcaggccuuauucagaucuaccggugguaccgccaccauggacuggaucuggagaauccuguuccuggugggggcugccacuggcgcccauagccagugcgugaaucugagaaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggacguuuauuaucauaagaacaacaagagcuggauggagagcggaguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcguccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccgcuacagacuguucagaaagagcaaucugaagccuuucgagagagacaucagcaccgagaucuaccaggccggcagcaagccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccagggcgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccucggggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcagaacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccugugacucgagacuaguagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - RNA, 4130 nt, Artificial sequence
  mRNA sequence of RBMRNA-Delta 3 vaccine
augagacaagcuugucccgcagucggcguccagcggcucugcuuguucgugugugugucguugcaggccuuauucagaucuaccggugguaccgccaccauguucguguuccuggugcugcugccccuggugagcagccagugcgugaaccugagaacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccuggacguguacuaccacaagaacaacaagagcuggauggagagcggaguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucaguccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccgcuaccggcuguuccggaagagcaaccugaagcccuucgagcgggacaucagcaccgagaucuaccaagccggcuccaagccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccagggcgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucacgcggcggcagcggcagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcgccaacagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcagaacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugacucgagacuaguagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - protein, 19 aa, Artificial sequence
  signal sequence
MDWIWRILFLVGAATGAHS

SEQ ID NO: 18 - protein, 27 aa, Artificial sequence
  Amino acid sequence of trimer domain
GYIPEAPRDGQAYVRKDGEWVLLSTFL

SEQ ID NO: 19 - protein, 4 aa, Artificial sequence
  linker sequence
SAIG

SEQ ID NO: 20 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGSGSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSANSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGSGSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSANSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1270 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGSGSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSANSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - DNA, 3813 bp, Artificial sequence
  DNA sequence of RBMRNA-Omicron vaccine
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgtcatcagcggcaccaatggcaccaagagattcgacaatcctgtgctgcctttcaatgacggcgtgtacttcgccagcatcgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggaccataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattatcgtgagagagccggaggacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcgacgaggtgttcaatgccaccagattcgccagcgtgtacgcatggaaccgcaagcggataagcaattgcgtggccgactacagcgtgctgtacaatctggccccattcttcaccttcaaatgttatggtgtttcgccaacaaagctgaatgacctgtgcttcaccaatgtgtacgccgacagcttcgtgatcagaggcgacgaggtgagacagatcgcgccagggcagaccggcaacatcgccgactacaattacaagctgcctgacgacttcaccggctgcgtgatcgcgtggaactctaacaagctggactcgaaagtttcaggcaattacaattacctgtacagactgttcagaaagagcaatctgaagcctttcgagagagacatcagcaccgagatctaccaggccggcaacaagccgtgtaatggcgtggcgggcttcaattgctacttccctctgcgcagctacagcttccggcctacctacggcgtgggccaccagccttacagagtggtggtgctgagcttcgagctgctgcacgctcccgctaccgtgtgcggccctaagaagagcaccaatctggtgaagaataagtgcgtgaatttcaatttcaatggtctaaagggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggcaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagtacgtgaataatagctacgagtgcgacatccctatcggcgccggcatctgcgccagctaccagacccagaccaagagccacggcggcagcggcagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaagagagccctgaccggcatcgccgtggagcaggacaagaatacccaggaggtgttcgcccaggtgaagcagatctacaagactccgccgatcaagtacttcggcggcttcaatttcagccaaatactcccagatccaagcaagcctagcgccaacagcttcatcgaggacctgctgttcaataaggtgaccctggccgacgccggcttcatcaagcagtacggcgactgcctaggtgatattgcggcaagagacctgatctgcgcccagaagtttaagggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcacaatgcccaggccctgaataccctggtgaagcagctgagcagcaagttcggcgccatcagtagtgtactcaacgacatcttcagcagactggacccaccagaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctaa

SEQ ID NO: 27 - RNA, 4127 nt, Artificial sequence
  mRNA sequence of RBMRNA-Omicron vaccine
augagacaagcuugucccgcagucggcguccagcggcucugcuuguucgugugugugucguugcaggccuuauucagaucuaccggugguaccgccaccauguucguguuccuggugcugcugccucuggugagcagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgucaucagcggcaccaauggcaccaagagauucgacaauccugugcugccuuucaaugacggcguguacuucgccagcaucgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggaccauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaucgugagagagccggaggaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucgacgagguguucaaugccaccagauucgccagcguguacgcauggaaccgcaagcggauaagcaauugcguggccgacuacagcgugcuguacaaucuggccccauucuucaccuucaaauguuaugguguuucgccaacaaagcugaaugaccugugcuucaccaauguguacgccgacagcuucgugaucagaggcgacgaggugagacagaucgcgccagggcagaccggcaacaucgccgacuacaauuacaagcugccugacgacuucaccggcugcgugaucgcguggaacucuaacaagcuggacucgaaaguuucaggcaauuacaauuaccuguacagacuguucagaaagagcaaucugaagccuuucgagagagacaucagcaccgagaucuaccaggccggcaacaagccguguaauggcguggcgggcuucaauugcuacuucccucugcgcagcuacagcuuccggccuaccuacggcgugggccaccagccuuacagagugguggugcugagcuucgagcugcugcacgcucccgcuaccgugugcggcccuaagaagagcaccaaucuggugaagaauaagugcgugaauuucaauuucaauggucuaaagggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccagggcgugaauugcaccgaggugccuguggccauccacgccgaccagcugacucccacuuggaggguauauuccacgggaagcaauguguuccagaccagagccggcugccugaucggcgccgaguacgugaauaauagcuacgagugcgacaucccuaucggcgccggcaucugcgccagcuaccagacccagaccaagagccacggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaagagagcccugaccggcaucgccguggagcaggacaagaauacccaggagguguucgcccaggugaagcagaucuacaagacuccgccgaucaaguacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaaggguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucacaaugcccaggcccugaauacccuggugaagcagcugagcagcaaguucggcgccaucaguaguguacucaacgacaucuucagcagacuggacccaccagaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugaccuccuguuguuccuguuugaaaggguguuguucguguggguccugcugcaaguucgacgaggacgacagcgagccugugcugaagggcgugaagcugcacuacaccuaacucgagacuaguagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - DNA, 6800 bp, Artificial sequence
  DNA sequence of plasmid template of RBMRNA-Omicron vaccine
tggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactataatgagacaagcttgtcccgcagtcggcgtccagcggctctgcttgttcgtgtgtgtgtcgttgcaggccttattcagatctaccggtggtaccgccaccatgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgtcatcagcggcaccaatggcaccaagagattcgacaatcctgtgctgcctttcaatgacggcgtgtacttcgccagcatcgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggaccataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattatcgtgagagagccggaggacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcgacgaggtgttcaatgccaccagattcgccagcgtgtacgcatggaaccgcaagcggataagcaattgcgtggccgactacagcgtgctgtacaatctggccccattcttcaccttcaaatgttatggtgtttcgccaacaaagctgaatgacctgtgcttcaccaatgtgtacgccgacagcttcgtgatcagaggcgacgaggtgagacagatcgcgccagggcagaccggcaacatcgccgactacaattacaagctgcctgacgacttcaccggctgcgtgatcgcgtggaactctaacaagctggactcgaaagtttcaggcaattacaattacctgtacagactgttcagaaagagcaatctgaagcctttcgagagagacatcagcaccgagatctaccaggccggcaacaagccgtgtaatggcgtggcgggcttcaattgctacttccctctgcgcagctacagcttccggcctacctacggcgtgggccaccagccttacagagtggtggtgctgagcttcgagctgctgcacgctcccgctaccgtgtgcggccctaagaagagcaccaatctggtgaagaataagtgcgtgaatttcaatttcaatggtctaaagggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggcaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagtacgtgaataatagctacgagtgcgacatccctatcggcgccggcatctgcgccagctaccagacccagaccaagagccacggcggcagcggcagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaagagagccctgaccggcatcgccgtggagcaggacaagaatacccaggaggtgttcgcccaggtgaagcagatctacaagactccgccgatcaagtacttcggcggcttcaatttcagccaaatactcccagatccaagcaagcctagcgccaacagcttcatcgaggacctgctgttcaataaggtgaccctggccgacgccggcttcatcaagcagtacggcgactgcctaggtgatattgcggcaagagacctgatctgcgcccagaagtttaagggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcacaatgcccaggccctgaataccctggtgaagcagctgagcagcaagttcggcgccatcagtagtgtactcaacgacatcttcagcagactggacccaccagaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctaactcgagactagtagccaacaccctgtctaaaaaacataaatttctttaatcattttgcctcttttctctgtgcttcaattaataaaaaatggaaagaacctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatcctctagactgcaggagctcgatatcgaattcgtattctatagtgtcacctaaatccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggggcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagct

SEQ ID NO: 29 - protein, 1273 aa, Unknown
  Amino acid sequence of SARS-CoV-2 wild type S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 1250 aa, Artificial sequence
  Amino acid sequence of recombinant S protein
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 31 - protein, 1231 aa, Artificial sequence
  Amino acid sequence of recombinant S protein
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 32 - protein, 1274 aa, Artificial sequence
  Amino acid sequence of recombinant S protein
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMSAIGGYIPEAPRDGQAYVRKDGEWVLLSTFL

SEQ ID NO: 33 - protein, 1255 aa, Artificial sequence
  Amino acid sequence of recombinant S protein
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMSAIGGYIPEAPRDGQAYVRKDGEWVLLSTFL

SEQ ID NO: 34 - RNA, 4085 nt, Artificial sequence
  mRNA sequence of recombinant S protein
aggagacaagcuugucccgcagucggcguccagcggcucugcuuguucgugugugugucguugcaggccuuauucagaucuaccggugguaccgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccugugacucgagacuaguagccaacacccugucuaaaaaacauaaauuucuuuaaucauuuugccucuuuucucugugcuucaauuaauaaaaaauggaaagaaccuggauccgucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 3973 nt, Artificial sequence
  mRNA sequence of recombinant S protein
gucccgcagucggcguccagcggcucugcuuguucgugugugugucguugcaggccuuauucgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccugugaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - RNA, 4157 nt, Artificial sequence
  mRNA sequence of recombinant S protein
aggagacaagcuugucccgcagucggcguccagcggcucugcuuguucgugugugugucguugcaggccuuauucagaucuaccggugguaccgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccugugacucgagacuaguagccaacacccugucuaaaaaacauaaauuucuuuaaucauuuugccucuuuucucugugcuucaauuaauaaaaaauggaaagaaccuggauccgucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 37 - RNA, 4100 nt, Artificial sequence
  mRNA sequence of recombinant S protein
aggagacaagcuugucccgcagucggcguccagcggcucugcuuguucgugugugugucguugcaggccuuauucagaucuaccggugguaccgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccugugacucgagacuaguagccaacacccugucuaaaaaacauaaauuucuuuaaucauuuugccucuuuucucugugcuucaauuaauaaaaaauggaaagaaccuggauccgucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - protein, 1266 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - protein, 1266 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - protein, 1266 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 1266 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 42 - protein, 1266 aa, Artificial sequence
  Amino acid sequence of recombinant S protein
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - RNA, 4115 nt, Artificial sequence
  mRNA sequence of RBMRNA-Lambda vaccine
augagacaagcuugucccgcagucggcguccagcggcucugcuuguucgugugugugucguugcaggccuuauucagaucuaccggugguaccgccaccauguucguguuccuggugcugcugccucuggugagcagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaaugucaucaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacaacucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccaguacagacuguucagaaagagcaaucugaagccuuucgagagagacaucagcaccgagaucuaccaggccggcagcacaccguguaauggcguggagggcuucaauugcuacuccccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccagggcgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugaacguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccaccagaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugaccuccuguuguuccuguuugaaaggguguuguucguguggguccugcugcaaguucgacgaggacgacagcgagccugugcugaagggcgugaagcugcacuacaccuaacucgagacuaguagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa